(19) United States
(12) Patent Application Publication
(10) Pub. No.: US 2021/0355539 A1
(43) Pub. Date: Nov. 18, 2021

(54) BIOMARKERS FROM MINIMALLY INVASIVE SAMPLING REFLECTIVE OF THE PLACENTAL IMMUNE MICROENVIRONMENT

(71) Applicant: NEW YORK UNIVERSITY, New York, NY (US)

(72) Inventor: Nazeeh Hanna, Dix Hills, NY (US)

(21) Appl. No.: 17/320,333

(22) Filed: May 14, 2021

Related U.S. Application Data
(60) Provisional application No. 63/025,410, filed on May 15, 2020.

(51) Int. Cl. C12Q 1/6883; C12Q 1/686

(52) U.S. Cl. CPC C12Q 1/6883; C12Q 2600/178; C12Q 1/686

(57) ABSTRACT

The present invention relates to minimally-invasive methods of measuring placenta-specific miRNA to assess immunotolerance during pregnancy. Disclosed are methods to diagnose and treat pathologic pregnancy based upon the altered level of placenta-specific miRNA in a pregnant mother.

### CROSS REFERENCE TO RELATED APPLICATIONS

This application claims priority to U.S. Provisional Application No. 63/025,410, filed May 15, 2020 which is hereby incorporated by reference herein in its entirety.

### BACKGROUND OF THE INVENTION

Pregnancy represents a unique challenge for the maternal-fetal immune interface, requiring the balance between immunosuppression, essential for the maintenance of semi-allogeneic fetus, and pro-inflammatory host defense (Kanellopoulos-Langevin, et al., 2003, Reproductive biology and endocrinology, 1:121; Gaunt and Ramin, 2001, Am J Perinatol, 18:299-312; Dekel et al., 2014, Am J Reprod Immunol, 72(2): 141-7). This balance is important in order to protect the mother and fetus from invading organisms. Adaptation to repeated inflammatory stimulation, with a controlled and downregulated TNF-α expression and inflammatory markers secretion, may be critical in preventing rejection of the fetus by the maternal immune system and in protecting the product of conception from excessive maternal inflammatory responses to infectious agents (DiGiulio et al., 2013, Psychosomatic Medicine, 75(7): 658-669; Blackburn and Loper, 1992, Maternal, fetal, and neonatal physiology: a clinical perspective. WB Saunders; Philadelphia; Stables, 1999, Physiology in Childbearing. Bailliere Tindall; Edinburgh). This adaptation (called immuno tolerance) is a well-described process in which cells exposed to repeated inflammatory stimuli become less responsive to subsequent exposures, thus suppressing an overly aggressive inflammatory response. Endotoxin tolerance is an example of immune tolerance and was initially described in 1946 by Beeson when he reported that repeated inoculation of rabbits with sub-lethal typhoid injections caused a significant reduction in the vaccine-induced fever (Beeson, 1946, Proc Soc Exp Biol Med, 61: 248-250). Lack of endotoxin tolerance leads to various pregnancy related pathologies including preterm birth (Marzi et al., 1996, Clinical and Experimental Immunology, 106(1): 127-133; Alijotas-Reig et al., 2014, Placenta, 35: 241-248; Dudley, 1997, Journal of Reproductive Immunology, 36(1-2): 93-109; Murphy et al., 2009, American Journal of Obstetrics and Gynecology, 200(3): 308; Wegmann et al., 1993, Immunol Today, 14(7): 343-35; Kim et al. 2018, Am J Reprod Immunol, December 26: e13080). To date, the exact mechanisms that contribute to the establishment and maintenance of tolerance are not completely understood.

Thus, there is a need in the art for improved compositions and methods for detecting and treating defective immune tolerance during pregnancy. This invention satisfies this unmet need.

### SUMMARY OF THE INVENTION

In one aspect, the present invention provides a method for diagnosing a subject as having, or being at risk for having, pathologic pregnancy. In one embodiment, the method comprises measuring the level of miRNA519c in a biological sample from the subject; and comparing the level of miRNA519c in the biological sample from the subject to the level of miRNA519c in a comparator, wherein a differential level of miRNA519c in the biological sample relative to the comparator indicates that the subject has, or is at risk for developing, pathologic pregnancy.

In one embodiment, the pathologic pregnancy is associated with the pathophysiology of inflammation. In one embodiment, the pathologic pregnancy is preterm premature rupture of membrane (PPROM), preterm labor, preeclampsia, intrauterine growth restriction, placental abruption, chromosomal anomalies or chorioamnionitis.

In one embodiment, the biological sample is at least one selected from the group consisting of saliva, urine, blood, serum, and plasma.

In one embodiment, measuring the level of miRNA519c in the biological sample from the subject comprises at least one technique selected from the group consisting of reverse transcription, polymerase chain reaction (PCR), and microarray analysis.

In one embodiment, the method comprises measuring the level of miRNA519c in extracellular vesicles (EVs) isolated from the biological sample.

In one embodiment, the method further comprises administering to the subject a therapeutic agent to treat or prevent pathologic pregnancy.

In one embodiment, the method further comprises measuring an increased level of one or more biomarkers associated with inflammation in the subject relative to a comparator.

In one embodiment, measurement of an increased level of one or more biomarkers associated with inflammation and the measurement of a differential level of miRNA519c indicates that the subject will proceed to delivery of the fetus or other pathologic pregnancy outcome.

In one embodiment, the method comprises administration to the subject of a specific therapy selected from: prenatal steroids, neuroprotective agents, and antibiotics.

In one aspect, the present invention provides a method of treating or preventing pathologic pregnancy in a subject. In one embodiment, the method comprises measuring the level of miRNA519c in a biological sample from the subject; comparing the level of miRNA519c in the biological sample from the subject to the level of miRNA519c in a comparator, wherein a differential level of miRNA519c in the biological sample relative to the comparator indicates that the subject has, or is at risk for developing, pathologic pregnancy; and administering to the subject a therapeutic agent to treat or prevent pathologic pregnancy.

In one embodiment, the therapeutic agent comprises an immunosuppressive agent. In one embodiment, the therapeutic agent comprises an agent that increases or decreases the expression or activity of miRNA519c in the placenta of the subject. In one embodiment, the therapeutic agent comprises miRNA519c or a miRNA519c mimic. In one embodiment, the therapeutic agent comprises a nucleic acid molecule encoding miRNA519c or a miRNA519c mimic.

In one aspect, the present invention provides a method of preparing a sample. In one embodiment, the method comprises providing a biological sample from a subject; isolating extracellular vesicles from the biological sample; selectively extracting RNA from the isolated extracellular vesicles; and performing an amplification reaction on the extracted RNA to detect the level of miRNA519c in the biological sample, wherein the amplification reaction is performed using primers specific for miRNA519c.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1C depict the results of example experiments demonstrating that endotoxin tolerance is a mechanism present in the placenta tissue.

FIG. 1A: Diagram of the experiment's timeline. LPS treatment was given after 24 or 48 hours of tissue culture and media was collected at day 1, 2 and 3 and analyzed for pro and anti-inflammatory mediators. Placenta tissues were collected after 3 days of culture. FIG. 1B and FIG. 1C: ELISA for TNF-α, IL-1β and IL10 in term (FIG. 1B; n=9) and second trimester (FIG. 1C; n=6) placenta explants media after being cultured and exposed to LPS showing a decrease of pro inflammatory interleukins after 2 LPS exposures compared to the level after only one exposure. FIG. 1D: MTT assay showing that tissue availability decreased after LPS treatment compared to medium but was constant after one or two doses of LPS.

FIG. 2A through FIG. 2E depict the results of example experiments demonstrating that extracellular vesicles (EVs) are endotoxin tolerance's mediators. FIG. 2A: Nanosight analysis showing the 2 method's results (ultracentrifugation-UC and kit) to isolate EVs: the kit obtained more EVs compared to the UC. Average size of the particles was 140-160 with the Kit and 120-140 with the UC. n=4. FIG. 2B: TNF-α expression after treatment with Cytocalasin D and LPS. Y axis: TNF-α concentration in pg/ml and X axis: treatment of the placental explants. Medium=blue column, treated with media in day 1 as well as day 2. LPS 1×=green column, media in day 1 and LPS in day 2. LPS2×=red column, LPS in day 1 and day 2. Cyt D=maroon column, cytocalasin D in day 1 and day 2. LPSX2/CytD: pink column, LPS and Cytocalasin-D in day 1 and day 2. *p<0.05 **p<0.001. TNF-α was decreased as expected after 2 doses of LPS compared to one dose of LPS. If treated with Cytocalasin-D, the TNF-α level was similar to the one after one dose of LPS treatment. n=9. FIG. 2C and FIG. 2D: Immunofluoresence of THP-1 cells (FIG. 2C, α-tubulin positive) and trophoblast (FIG. 2D, cytokeratin 7 positive) following 1.5 h incubation with PKH26 (red color) labeled extracellular vesicles. Red vesicles (white arrows) are visibly localized near the cell surface and within the cytoplasm. Blue: DAPI. Scale: 10 nm. FIG. 2E: TNF-α measured by ELISA showed a statistically significant decreased level in the media of THP-1 cells treated with LPS and co-cultured with EVs isolated from media of tollerized (LPS EVs) compared to the THP-1 cells treated with EVs from untreated placentas (CTL EVs). Y axis: TNF-α concentration in ng/ml and X axis: treatment of the THP-1 cells. n=7.

FIG. 3A through FIG. 3C depict the results of example experiments demonstrating that extracellular vesicles contain several microRNAs including miR-519c, a possible mediator of Endotoxin tolerance. FIG. 3A: RT-qPCR for miRNAs in placental EVs after LPS treatment compared to control. Data expressed as fold change. The 3 most abundant miRNAs in the extra cellular vesicles were miRNA-543, miRNA-519c and miRNA-145. FIG. 3B: miRNA 519c ISH signal is seen in placental tissue (green staining, FIG. 3B(a)) whereas no ISH signal is obtained with scramble probe (FIG. 3B(b)). Cells were labeled with PALP (red staining), marker for trophoblast. FIG. 3C: q-PCR and ELISA for TNF-α after transfection in THP-1 cells and primary trophoblast of miRNA-519c mimic and control and LPS challenge 24 hours later. miRNA-519c mimic transfection statistically significantly decreased TNF-α gene expression and protein production in both of the cells type compared to control mimic transfection.

FIG. 4A through FIG. 4D depict the results of example experiments demonstrating that miRNA-519c is transported to the target cells inside the extracellular vesicles. FIG. 4A: Representation of molecular mechanisms underlying miRNA-519c production in producing cells with EVs production and miRNA-519c release in the surrounding environment. FIG. 4B: qPCR for miRNA-519 precursors, miRNA-519c mature form at the level of the tissue and at the level of the EVs at different time points (3, 24 and 48 hours) showing that only the mature form inside the EVs increased over time. FIG. 4C: qPCR for miRNA-519c detecting increased miRNA-519c at the level of the media and EVs but not in the tissue after LPS induction. FIG. 4D: qPCR before and after 18 hours centrifugation of the media showing that after the ultracentrifugation (and the consequent removal of close to 100% of the EVs based on the work published by Shelke Gin 2014 (59)) confirming that the miRNA519c is mainly present inside EVs and in a minor quantity as free miRNA.

FIG. 5A through FIG. 5C depict the results of example experiments demonstrating that miRNA519c mediates endotoxin tolerance via PDE3B. FIG. 5A: Transfection of miRNA-519c mimic in trophoblast cells showed a statistically significant decrease of PDE3B mRNA level via q-PCR, n=7. FIG. 5B: TNF-α mRNA level before and after LPS exposure in siPDE3B transfected PTH and THP-1 cells: in both of the type of the cells, lack of PDE3B had an effect in TNF-α production during inflammation and during a steady state condition. FIG. 5C: Representation of molecular mechanisms underlying miRNA-519c action in target cells with inhibition of PDE3B resulting in decrease of TNF-α production.

FIG. 6A through FIG. 6D depict the results of example experiments demonstrating that miRNA-519c decreases during pregnancy and its levels are decreased in mothers with inflammatory processes. FIG. 6A: qPCR for miRNA-519c showing that its levels were stable during pregnancy in placental tissue. Data from first and second trimester elective terminations and from term CS not in labor. FIG. 6B: miRNA-519c level in placentas from women affected by PEC (black pattern) was not different compared to women without it. FIG. 6C: miRNA-519c level in placentas from women undergoing inflammatory processes (such as Premature rupture of membrane or chorioamnionitis) was statistically significantly decreased compared to control (women not affected by any of those pathologies) confirming the pivotal role of miRNA-519c in controlling inflammation during pregnancy. FIG. 6D: miRNA-519c level in placentas from women at term and in labor was decreased (statistically significantly) compared to women at term, not in labor and who delivered via CS.

FIG. 7 depicts the results of example experiments demonstrating that TLR4 is not involved in the decreased TNF-α levels during Endotoxin Tolerance. Real Time PCR for TLR4 showing that repeated LPS treatment does not affect TLR4 level confirming that TNF-α decreased level during endotoxin tolerance is not TLR4 dependent.

FIG. 8 depicts the results of example experiments investigating the effect of transfection of several miRNAs in THP-1 cells. Transfection of different miRNAs in THP-1 cells showed that only miRNA-519c inhibited TNF-α secretion. N=4.

FIG. 9 depicts the results of example experiments examining the effect of transfection of miRNA-519c in THP-1 cells. Transfection of miRNA-519c mimic in THP-1 cells did not alter TLR4 gene expression proving that the decrease of TNF-α was not due to an increase TLR4 level n=3 p=0.062, not statistically significant.

FIG. 10 depicts the results of example experiments investigating the level of miR-519c in EVs from maternal plasma, amniotic fluid, and saliva. miR-519c had comparable expression in maternal plasma and amniotic fluid, and surprisingly higher expression in the saliva samples. Furthermore, EVs miR-519c expression was strongly correlated between the placenta and maternal plasma samples (n=8, Spearman r=0.96, p-value=0.0011), as well as between the placenta and saliva samples (n=8, Spearman r=0.74, p-value=0.046). However, there was no correlation between the placenta and amniotic fluid samples.

FIG. 11 depicts the results of example experiments demonstrating that the level of miR-519c is increased in pregnant mothers who are asymptomatic COVID-19 positive. This indicates that COVID-19 placental infection will increase miR-519c that will be protective against Infection-mediated preterm births.

FIG. 12 depicts the percent change in preterm infants (<28 weeks) in 2020 vs. 2019 in various neonatal intensive care units (NICUs) in the New York city area, indicating that COVID-19 infection was associated with a decrease in preterm births less than 28 weeks (often associated with infection-mediated preterm births).

### DETAILED DESCRIPTION

The present invention relates to compositions and methods for detecting and treating pathologic pregnancy and preterm labor. The present invention is based upon the discovery that miRNA519c (also referred to herein as miR-519c or miRNA-519c), a placenta-specific miRNA, plays a role in mediating immunotolerance in a pregnant mother, a vital mechanism for maintenance of the fetus. Further, it is discovered herein that the level of miRNA519c in non-invasive body samples from them other, is correlated to the level in the placenta, thereby allowing for non-invasive assessment of immunotolerance and diagnosis of pathologic pregnancy and preterm labor.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “antibody,” as used herein, refers to an immunoglobulin molecule which is able to specifically bind to a specific epitope of a binding partner molecule. Antibodies can be intact immunoglobulins derived from natural sources, or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” or “binding fragment” refers to at least one portion of an antibody and refers to the antigenic determining variable regions of an intact antibody. Examples of antibody fragments include, but are not limited to, Fab, Fab′, F(ab′)2, and Fv fragments, linear antibodies, sdAb (either VL or VH), camelid VHH domains, scFv antibodies, and multi-specific antibodies formed from antibody fragments. The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it was derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

“Antisense,” or “antisense nucleic acid molecule,” as used herein, refers to a nucleic acid comprising a sequence which is complementary to a target sequence, such as, by way of example, complementary to a target miRNA sequence, including, but not limited to, a mature target miRNA sequence, or a related sequence thereof. Typically, an antisense sequence is fully complementary to the target sequence across the full length of the antisense nucleic acid sequence.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, cervical secretions, vaginal secretions, phlegm and sputum. A body fluid sample may be collected by any suitable method. The body fluid sample may be used immediately or may be stored for later use. Any suitable storage method known in the art may be used to store the body fluid sample: for example, the sample may be frozen at about −20° C. to about −70° C. Suitable body fluids are acellular fluids. “Acellular” fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells however, some may contain cell fragments or cellular debris. Examples of acellular fluids include plasma or serum, or body fluids from which cells have been removed.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes, examination of cells, cytogenetics, and immunophenotyping of blood cells.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60% or at least about 80% of corresponding positions in each of the molecules are occupied by nucleotides which normally base pair with each other (e.g., A:T and G:C nucleotide pairs).

As used herein, “conjugated” refers to covalent attachment of one molecule to a second molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

The term “comparator” describes a material comprising none, or a normal, low, or high level of one of more of the marker (or biomarker) expression products of one or more the markers (or biomarkers) of the invention, such that the comparator may serve as a control or reference standard against which a sample can be compared.

As used herein, the term “diagnosis” means detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other diagnosing and/or staging methods known in the medical art for a particular disease or disorder.

As used herein, the phrase “difference of the level” refers to differences in the quantity of a particular marker, such as a nucleic acid (e.g., microRNA, etc.) or a protein, in a sample as compared to a control or reference level. For example, the quantity of a particular biomarker may be present at an elevated amount or at a decreased amount in samples of patients with a disease compared to a reference level. In one embodiment, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “dysregulated” and “dysregulation” as used herein describes a decreased (down-regulated) or increased (up-regulated) level of expression of a miRNA present and detected in a sample obtained from subject as compared to the level of expression of that miRNA in a comparator sample, such as a comparator sample obtained from one or more normal, not-at-risk subjects, or from the same subject at a different time point. In some instances, the level of miRNA expression is compared with an average value obtained from more than one not-at-risk individuals. In other instances, the level of miRNA expression is compared with a miRNA level assessed in a sample obtained from one normal, not-at-risk subject.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” includes determining the amount of something present, as well as determining whether it is present or absent.

“Differentially increased expression” or “up regulation” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments there between compared to a comparator.

“Differentially decreased expression” or “down regulation” refers to expression levels which are at least 10% or more, for example, 20%, 30%, 40%, or 50%, 60%, 70%, 80%, 90% lower or less, and/or 2.0 fold, 1.8 fold, 1.6 fold, 1.4 fold, 1.2 fold, 1.1 fold or less lower, and any and all whole or partial increments there between compared to a comparator.

As used herein “endogenous” refers to any material from or produced inside an organism, cell, tissue or system.

As used herein “exogenous” refers to any material from or produced outside an organism, cell, tissue or system.

The term “expression” as used herein is defined as the transcription and/or translation of a particular nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.”

“Inhibitors,” “activators,” and “modulators” of the markers are used to refer to activating, inhibiting, or modulating molecules, respectively, identified using in vitro and in vivo assays of autoimmune disease biomarkers. Inhibitors are compounds that, e.g., bind to, partially or totally block activity, decrease, prevent, delay activation, inactivate, desensitize, or down regulate the activity or expression of autoimmune disease biomarkers. “Activators” are compounds that increase, open, activate, facilitate, enhance activation, sensitize, agonize, or up regulate activity of autoimmune disease biomarkers. Agonists, inhibitors, activators, or modulators also include genetically modified versions of autoimmune disease biomarkers, e.g., versions with altered activity, as well as naturally occurring and synthetic ligands, antagonists, agonists, antibodies, peptides, cyclic peptides, nucleic acids, antisense molecules, ribozymes, RNA interference (RNAi), microRNA, and small interfering RNA (siRNA) molecules, small organic molecules and the like. Such assays for inhibitors and activators include, e.g., expressing autoimmune disease biomarkers in vitro, in cells, or cell extracts, applying putative modulator compounds, and then determining the functional effects on activity, as described elsewhere herein.

The term “inhibit,” as used herein, means to suppress or block an activity or function, for example, about ten percent relative to a control value. For example, the activity is suppressed or blocked by 50% compared to a control value, or by 75%, or by 95%. “Inhibit,” as used herein, also means to reduce the level of a molecule, a reaction, an interaction, a gene, a miRNA, an mRNA, and/or a protein's expression, stability, amount, function or activity by a measurable amount or to prevent production entirely. Inhibitors are compounds that, e.g., bind to, partially or totally block activity, decrease, prevent, delay activation, inactivate, desensitize, or down regulate a protein, a gene, a miRNA, and an mRNA stability, expression, function and activity, e.g., antagonists.

As used herein, an “instructional material” includes a publication, a recording, a diagram, or any other medium of expression which can be used to communicate the usefulness of a compound, composition, vector, method or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

As used herein, “isolated” means altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

The term “vector,” as used in the context of the present invention, refers to a DNA molecule used as a vehicle to carry genetic material into a cell or other host, where it can be replicated and/or expressed.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

As used herein, “microRNA” or “miRNA” describes small non-coding RNA molecules, generally about 15 to about 50 nucleotides in length, for example, 17-23 nucleotides in length, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes.

A “mutation,” as used herein, refers to a change in nucleic acid or polypeptide sequence relative to a reference sequence (which may be a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

As used herein, the term “neutralizing” may refer to neutralization of biological activity of a target when a binding molecule specifically binds the target. In the context of miRNAs, a neutralizing binding molecule may be a small molecule, a ribozyme, a miRNA, an antisense nucleic acid molecule, a polypeptide, or another type of molecule, the binding of which to the miRNA results in inhibition of a biological activity of the miRNA. For example, the neutralizing binding molecule binds one or more miRNAs and reduces a biological activity of the miRNAs by at least about 20%, 40%, 60%, 80%, 85% or more.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means. “Polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target RNA species. Consequently, in some instances, hammerhead-type ribozymes are generally superior to tetrahymena-type ribozymes for inactivating specific RNA species, and 18-base recognition sequences are superior compared to shorter recognition sequences which may occur randomly within various unrelated RNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

The term “RNA” as used herein is defined as ribonucleic acid.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “DNA” as used herein is defined as deoxyribonucleic acid.

The term “recombinant DNA” as used herein is defined as DNA produced by joining pieces of DNA from different sources.

The term “recombinant polypeptide” as used herein is defined as a polypeptide produced by using recombinant DNA methods.

By “pharmaceutically acceptable” it is meant, for example, a carrier, diluent or excipient that is compatible with the other ingredients of the formulation and generally safe for administration to a recipient thereof. As used herein, “pharmaceutically acceptable carrier” includes any material, which when combined with the conjugate retains the conjugates' activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically, such carriers contain excipients such as starch, milk, sugar, some types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The terms “modulator” and “modulation” of a molecule of interest, as used herein in its various forms, is intended to encompass antagonism, agonism, partial antagonism and/or partial agonism of an activity associated with the molecule of interest. The terms “modulator” and “modulation” are used interchangeably with the terms “regulator” and “regulation,” respectively. In various embodiments, “modulators” may inhibit or stimulate molecule expression or activity. Such modulators include small molecule agonists and antagonists of a molecule, i.e., antagomirs, antisense molecules, ribozymes, miRNAs, triplex molecules, and RNAi polynucleotides, and others.

As used herein, the term “providing a prognosis” refers to providing a prediction of the probable course and outcome of a disease or disorder, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

“Sample” or “biological sample” as used herein means a biological material isolated from an individual. The biological sample may contain any biological material suitable for detecting the desired biomarkers, and may comprise cellular and/or non-cellular material obtained from the individual.

By the term “specifically binds,” as used herein with respect to an antibody, is meant an antibody which recognizes a specific binding partner molecule, but does not substantially recognize or bind other molecules in a sample. For example, an antibody that specifically binds to a binding partner molecule from one species may also bind to that binding partner molecule from one or more species. But, such cross-species reactivity does not itself alter the classification of an antibody as specific. In another example, an antibody that specifically binds to binding partner molecule may also bind to different allelic forms of the binding partner molecule. However, such cross reactivity does not itself alter the classification of an antibody as specific.

In some instances, the terms “specific binding” or “specifically binding”, can be used in reference to the interaction of an antibody, a protein, or a peptide with a second binding partner molecule, to mean that the interaction is dependent upon the presence of a particular structure (e.g., an antigenic determinant or epitope) on the binding partner molecule; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. In some instances, the terms “specific binding” and “specifically binding” refers to selective binding, wherein the antibody recognizes a sequence or conformational epitope important for the enhanced affinity of binding to the binding partner molecule.

“Standard control value” as used herein refers to a predetermined amount of a particular protein or nucleic acid that is detectable in a biological sample. The standard control value is suitable for the use of a method of the present invention, in order for comparing the amount of a protein or nucleic acid of interest that is present in a biological sample. An established sample serving as a standard control provides an average amount of the protein or nucleic acid of interest in the biological sample that is typical for an average, healthy person of reasonably matched background, e.g., gender, age, ethnicity, and medical history. A standard control value may vary depending on the protein or nucleic acid of interest and the nature of the sample (e.g., serum).

The terms “subject,” “patient,” “individual,” and the like are used interchangeably herein, and refer to any animal, or cells thereof whether in vitro or in situ, amenable to the methods described herein. Thus, the individual may include, for example, dogs, cats, pigs, cows, sheep, goats, horses, rats, monkeys, and mice and humans. In some non-limiting embodiments, the patient, subject or individual is a human.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

A “therapeutic” treatment is a treatment administered to a subject who exhibits signs of pathology, for the purpose of diminishing or eliminating those signs.

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

The terms “treat,” “treating,” and “treatment,” refer to therapeutic or preventative measures described herein. The methods of “treatment” employ administration to a subject, in need of such treatment, a composition of the present invention, for example, a subject afflicted a disease or disorder or a subject who ultimately may acquire such a disease or disorder in order to prevent, cure, delay, reduce the severity of, or ameliorate one or more symptoms of the disorder or recurring disorder, or in order to prolong the survival of a subject beyond that expected in the absence of such treatment.

The terms “underexpress,” “underexpression,” “underexpressed,” or “down-regulated” interchangeably refer to a protein or nucleic acid that is transcribed or translated at a detectably lower level in a biological sample from a woman with autoimmune disease, in comparison to a biological sample from a woman without autoimmune disease. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using conventional techniques for detecting mRNA (i.e., Q-PCR, RT-PCR, PCR, hybridization) or proteins (i.e., ELISA, immunohistochemical techniques). Underexpression can be 10%, 20%, 30%, 40%, 50%, 60%, 70%, 80%, 90% or less in comparison to a control. In some instances, underexpression is 1-, 2-, 3-, 4-, 5-, 6-, 7-, 8-, 9-, 10-fold or more lower levels of transcription or translation in comparison to a control.

The terms “overexpress,” “overexpression,” “overexpressed,” or “up-regulated” interchangeably refer to a protein or nucleic acid (RNA) that is transcribed or translated at a detectably greater level, usually in a biological sample from a woman with autoimmune disease, in comparison to a biological sample from a woman without autoimmune disease. The term includes overexpression due to transcription, post transcriptional processing, translation, post-translational processing, cellular localization (e.g., organelle, cytoplasm, nucleus, cell surface), and RNA and protein stability, as compared to a cell from a woman without autoimmune disease. Overexpression can be detected using conventional techniques for detecting mRNA (i.e., Q-PCR, RT-PCR, PCR, hybridization) or proteins (i.e., ELISA, immunohistochemical techniques). Overexpression can be 10%, 20%, 30%, 40%, 50%, 60%, 70%, 80%, 90% or more in comparison to a cell from a woman without autoimmune disease. In some instances, overexpression is 1-, 2-, 3-, 4-, 5-, 6-, 7-, 8-, 9-, 10-fold, or more higher levels of transcription or translation in comparison to a cell from a woman without autoimmune disease.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention relates to the finding that a placenta-specific miRNA, miRNA519c, plays a role placental function to maintain healthy pregnancy, including playing a role in protective immunotolerance in pregnant mothers. Further, it is demonstrated herein that the level miRNA519c can be detected in non-invasive body samples, including, but not limited to, saliva, blood, cervical secretions, vaginal secretions, urine and amniotic fluid, to determine if a pregnant mother at risk for pathologic pregnancy and preterm labor. In some embodiments, the pathologic pregnancy is preterm premature rupture of membrane (PPROM), preterm labor, preeclampsia, intrauterine growth restriction, placental abruption, chromosomal anomalies or chorioamnionitis

In some embodiments, the level of miRNA519c, alone or in combination with markers of inflammation, are used to determine if a pregnant mother is at risk for pathologic pregnancy or preterm labor. Other markers include, but are not limited to, other miRNAs or inflammatory markers such as cytokines or chemokines. The miRNA519c, alone or in combination with other miRNAs or inflammatory markers, can be measured in a single sample, such as saliva, or in a combination of several samples, such as saliva, urine, blood, or other body samples.

In one aspect, the methods generally provide for the detection, measuring, and comparison of a pattern of miRNA519c in a body sample from the pregnant mother. In certain embodiments, the method provides a non-invasive method of detecting the level of miRNA519c to assess the risk of pathologic pregnancy or preterm labor. For example, in one embodiment, the level of miRNA519c is measured in a saliva sample of the pregnant mother, which is found herein to be correlated to the level in the placenta.

In some embodiments, a change (e.g., a decrease or increase) in the level of miRNA519c in the sample obtained from the subject, relative to the level miRNA519c in a control sample, is indicative of deficient immunotolerance indicating that the subject has, or is at risk for having, pathologic pregnancy or preterm labor. For example, in one embodiment, the level of miRNA519c in the test sample is less than the level of miRNA519c in the control sample.

In some embodiments, the method indicates a subject's responsiveness to a treatment or therapy regimen. For example, in one embodiment, an increase in the level of miRNA519c in a test sample from the subject undergoing a treatment or therapy regiment, relative to a control sample, such as a sample obtained from the subject at an earlier time point, indicates that subject is responsive to the treatment or therapy regimen. In one embodiment, a decrease in the level of miRNA519c in a test sample from the subject undergoing a treatment or therapy regiment, relative to a control sample, such as a sample obtained from the subject at an earlier time point, indicates that subject is not responsive to the treatment or therapy regimen. In one embodiment, detecting no change in the level of miRNA519c in a test sample from the subject undergoing a treatment or therapy regiment, relative to a control sample, such as a sample obtained from the subject at an earlier time point, indicates that subject is not responsive to the treatment or therapy regimen.

Additional diagnostic markers may be combined with the miRNA519c level to construct models for predicting the presence or absence or stage of a disease. For example, clinical factors of relevance to the diagnosis of pathologic pregnancy or preterm labor include, but are not limited to, the subject's age, the subject's medical history, the subject's ethnicity, a physical examination, and other biomarkers.

Generally, the methods of this invention find use in diagnosing or for providing a prognosis for pathologic pregnancy by detecting the expression levels of biomarkers, which are differentially expressed (up- or down-regulated) in a sample from a subject. These markers can be used to distinguish the severity of pathologic pregnancy, or to assess the likelihood of pathologic pregnancy. These markers can also be used to provide a prognosis for the course of treatment in a subject with pathologic pregnancy In some embodiments, these markers can be used to distinguish subjects who will progress towards delivery of the fetus. For example, in one embodiment, the method comprises detecting a decreased level of miRNA519c in a sample from a subject, relative to a control sample, thereby indicating that the subject will progress towards active labor and delivery. For example, in some instances the method aids in decision making on whether to admit the subject for therapy/observation, transfer to a higher level facility, or discharge the subject to her home. If, using the described method, the subject is determined to be at risk for adverse pregnancy outcome, such as preterm labor, the method may further comprise administering specific therapies, including, but not limited to, prenatal steroids, neuroprotective agents, and antibiotics, to the subject.

In one embodiment, the methods of the present invention find use in assigning treatment to a subject having, or being of at risk for having, pathologic pregnancy or preterm labor. By detecting the expression levels of biomarkers found herein, the appropriate treatment can be assigned to a subject. In one embodiment, the treatment comprises administration of an agent that increases the expression or activity of miRNA519c. For example, in one embodiment, the treatment comprises administration of miRNA519c or miRNA519c mimic, or nucleic acid molecule encoding the same.

Diagnostic and prognostic kits comprising one or more markers for use are provided herein. Also provided by the invention are methods for identifying compounds that are able to prevent or treat pathologic pregnancy or preterm labor by modulating the expression level or activity of miRNA519c. Therapeutic methods are provided, wherein pathologic pregnancy or preterm labor treated or prevented using an agent that targets the markers of the invention.

In various embodiments, the methods of the invention relate to methods of assessing a subject's risk of having or developing pathologic pregnancy or preterm labor, methods of assessing the severity of a subject's pathologic pregnancy or preterm labor, methods of diagnosing pathologic pregnancy or preterm labor, methods of characterizing pathologic pregnancy or preterm labor, and methods of stratifying a subject having an pathologic pregnancy or preterm labor in a clinical trial.

In various embodiments of the compositions and methods of the invention described herein, the miRNA associated with pathologic pregnancy or preterm labor is miRNA519c. Sequences of miRNA519c are publicly available from miRbase (www.mirbase.org).

In some embodiments, the biomarkers of the invention are one or more miRNA associated with pathologic pregnancy or preterm labor which are down-regulated, or expressed at a lower than normal level in the disease state. For example, it is described herein that miRNA519c is down regulated or expressed at a lower than normal level in subjects having, or at risk for having, pathologic pregnancy or preterm labor. Thus, in some embodiments, the invention relates to compositions and methods useful for the diagnosis, assessment, and characterization of pathologic pregnancy or preterm labor in a subject in need thereof, based upon the detection of a decreased level of miR519c.

In one embodiment, the invention provides a method for detecting a marker of pathologic pregnancy or preterm labor. In one embodiment, the invention provides a method for monitoring the levels of miRNAs in response to treatment. In one embodiment, the invention provides a method for monitoring the level of miRNA519c.

In one embodiment, a treatment-induced change in miRNA519c is indicative of treatment efficacy or that a subject is responsive to treatment.

For example, in certain embodiments where a decrease in miRNA519c is associated with a pathologic pregnancy, a treatment-induced increase in miRNA519c is indicative of treatment efficacy or that a subject is responsive to treatment. Further, in certain embodiments where a decrease in miRNA519c is associated with a pathologic pregnancy, a treatment-induced decrease in miRNA519c is indicative that the subject is not responsive to treatment. Additionally, in certain embodiments where a decrease in miRNA519c is associated with a pathologic pregnancy, a lack of a treatment-induced increase in miRNA519c is indicative that the subject is not responsive to treatment.

For example, in certain embodiments where an increase in miRNA519c is associated with a pathologic pregnancy, a treatment-induced decrease in miRNA519c is indicative of treatment efficacy or that a subject is responsive to treatment. Further, in certain embodiments where an increase in miRNA519c is associated with a pathologic pregnancy, a treatment-induced increase in miRNA519c is indicative that the subject is not responsive to treatment. Additionally, in certain embodiments where an increase in miRNA519c is associated with a pathologic pregnancy, a lack of a treatment-induced decrease in miRNA519c is indicative that the subject is not responsive to treatment.

The miRNAs may be detected and measured using any methods presented herein, including but not limited to hybridization assays, direct sequencing methods, and array-based methods. The miRNAs may be reverse-transcribed and amplified by polymerase chain reaction, using a detectable reporter molecule for measuring amounts of the miRNAs in the biological sample. These assays are known in the art. In certain embodiments, the miRNA is detected outside or inside extracellular vesicles (EVs) isolated or purified from a body sample.

Accordingly, the invention provides a new and convenient platform for detecting a marker of pathologic pregnancy or preterm labor. In one embodiment, the system of the invention provides a platform for detecting a marker of pathologic pregnancy or preterm labor with at least 80% sensitivity, at least 90%, at least 91%, at least 92%, at least 93%, at least 94%, at least 95%, at least 96%, at least 97%, at least 98%, at least 99%, or at least 100%.

In one embodiment, the invention provides a system for detecting a marker of pathologic pregnancy or preterm labor, with at least 90%, 91%, 92%, 93%, 94%, 95%, 96%, 97%, 98%, 99%, 100% sensitivity; at least 90%, 91%, 92%, 93%, 94%, 95%, 96%, 97%, 98%, 99%, 100% specificity; or both at least 90%, 91%, 92%, 93%, 94%, 95%, 96%, 97%, 98%, 99%, 100% sensitivity and specificity. In one embodiment, the invention provides a system for detecting a marker of pathologic pregnancy or preterm labor with at least 90%, 91%, 92%, 93%, 94%, 95%, 96%, 97%, 98%, 99%, 100% accuracy.

### Sample Preparation

Test samples of acellular body fluid or cell-containing samples may be obtained from an individual, subject, or patient. Methods of obtaining test samples are well-known to those of skill in the art. Samples may include, but are not limited to, saliva, whole blood, serum, plasma, amniotic fluid, cervical secretions, vaginal secretions, cerebrospinal fluid (CSF), pericardial fluid, pleural fluid, synovial fluid, urine, and eye fluid. In some embodiments in which the test sample contains cells, the cells may be removed from the liquid portion of the sample by methods known in the art (e.g., centrifugation) to yield acellular body fluid.

In suitable embodiments, serum or plasma are used as the acellular body fluid sample. Plasma and serum can be prepared from whole blood using suitable methods well-known in the art. In these embodiments, data may be normalized by volume of acellular body fluid.

In some embodiments, the sample is processed to isolate extracellular vesicles. Extracellular vesicles can be isolated from the sample using techniques known in the art, including, but not limited to differential ultracentrifugation, density gradient centrifugation, precipitation, exclusion chromatography, and immunoaffinity capture-based isolation. In one embodiment, extracellular vesicles are isolated from the sample using a commercially available kit or reagent, including, but not limited to, Total Exosome Isolation Reagent (TEIR) (Invitrogen), Capturem Extracellular Vesicle Isolation Kit (Takara), ExoQuick (System biosciences), and exoEasy (Qiagen).

Variability in sample preparation of cell-containing samples can be corrected by normalizing the data by, for example, protein content or cell number. In some embodiments, the sample may be normalized relative to the total protein content in the sample. Total protein content in the sample can be determined using standard procedures, including, without limitation, Bradford assay and the Lowry method. In other embodiments, the sample may be normalized relative to cell number.

In one aspect, the present invention relates to a method of preparing a sample. In certain embodiments, the method comprises preparing a miRNA sample. In one embodiment, the method comprises providing a biological sample obtained from the subject. The biological sample can be a sample from any source, such as a body fluid (e.g., saliva, blood, plasma, serum, synovial fluid, cervical secretions, vaginal secretions, urine, amniotic fluid, etc.), or a tissue, or an exosome, or a cell, or a combination thereof. A biological sample can be obtained by appropriate methods, such as, by way of examples, biopsy or fluid draw. In one embodiment, the subject is a pregnant mother. In one embodiment, the subject is a pregnant mother having, or at risk for having, a pathological pregnancy or preterm labor. In one embodiment, the method comprises selectively isolating extracellular vesicles from the biological sample. In one embodiment, the method comprises isolating or extracting RNA from the biological sample or the isolated extracellular vesicles. For example, in one embodiment, the method comprises selectively extracting RNA from the biological sample or the isolated extracellular vesicles. In one embodiment, the method comprises performing an assay on the extracted RNA. In one embodiment, the method comprises amplifying one or more miRNA, such as miR-519c, from the isolated or extracted RNA. For example, in one embodiment, the method comprises performing an amplification reaction on the isolated or extracted RNA in order to detect one or more miRNA, such as miR-519c, where the amplification reaction is performed using primers specific for the one or more miRNA, such as primers specific for miR-519c.

### Assays

The present invention relates to the discovery that the expression level of placenta-specific miRNA519c is associated with the presence, risk, development, progression and severity of pathologic pregnancy or preterm labor. In various embodiments, the invention relates to a screening assay of a subject to determine the level of expression of miRNA519c associated with pathologic pregnancy or preterm labor in the subject. The present invention provides methods of assessing level of miRNA519c associated with pathologic pregnancy or preterm labor, as well as methods of diagnosing a subject as having, or as being at risk of developing, pathologic pregnancy or preterm labor based upon the level of expression of miRNA519c. In some embodiments, the diagnostic assays described herein are in vitro assays.

In one embodiment, the method of the invention is a diagnostic assay for assessing the presence, risk, development, progression and severity pathologic pregnancy or preterm labor in a subject in need thereof, by determining whether the level of miRNA519c is increased or decreased in a biological sample obtained from the subject. In various embodiments, to determine whether the level of the miRNA519c is increased or decreased in a biological sample obtained from the subject, the level of miRNA519c is compared with the level of at least one comparator control, such as a positive control, a negative control, a normal control, a wild-type control, a historical control, a historical norm, or the level of another reference molecule in the biological sample. In some embodiments, the diagnostic assay of the invention is an in vitro assay. In other embodiments, the diagnostic assay of the invention is an in vivo assay.

In various embodiments of the assays of the invention, the level of miRNA519c is determined to be down-regulated when the level of miRNA519c is decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In various embodiments of the assays of the invention, the level of miRNA519c is determined to be up-regulated when the level of miRNA519c is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, or by at least 5000%, when compared with a comparator control.

In the assay methods of the invention, a test biological sample from a subject is assessed for the expression level of miRNA519c. The test biological sample can be an in vitro sample or an in vivo sample. In various embodiments, the subject is a human subject, and may be of any race and age. In various embodiments, the subject is a pregnant woman, and may be of any stage of pregnancy. Representative subjects include those who are suspected of having pathologic pregnancy or preterm labor, those who have been diagnosed with pathologic pregnancy or preterm labor, those whose have pathologic pregnancy or preterm labor, those who have had pathologic pregnancy or preterm labor, those who at risk of a recurrence of pathologic pregnancy or preterm labor, and those who are at risk of developing pathologic pregnancy or preterm labor.

In some embodiments, an miRNA519c-binding molecule is used in vivo for the diagnosis of pathologic pregnancy or preterm labor. In some embodiments, the miRNA519c-binding molecule is nucleic acid that hybridizes with miRNA519c.

In one embodiment, the test sample is a sample containing at least a fragment of a nucleic acid comprising miRNA519c. The term, “fragment,” as used herein, indicates that the portion of a nucleic acid (e.g., DNA, mRNA or cDNA) that is sufficient to identify it as comprising miRNA519c.

In some embodiments, the test sample is prepared from a biological sample obtained from the subject. The biological sample can be a sample from any source which contains a nucleic acid comprising miRNA519c, such as a body fluid (e.g., saliva, blood, plasma, serum, cervical secretions, vaginal secretions, synovial fluid, urine, amniotic fluid, etc.), or a tissue, or an exosome, or a cell, or a combination thereof. A biological sample can be obtained by appropriate methods, such as, by way of examples, biopsy or fluid draw. The biological sample can be used as the test sample; alternatively, the biological sample can be processed to enhance access to polypeptides, nucleic acids, or copies of nucleic acids (e.g., copies of nucleic acids comprising miRNA519c), and the processed biological sample can then be used as the test sample. For example, in various embodiments, nucleic acid is prepared from a biological sample, for use in the methods. Alternatively, or in addition, if desired, an amplification method can be used to amplify nucleic acids comprising all or a fragment of a nucleic acid in a biological sample, for use as the test sample in the assessment of the expression level of miRNA519c.

The test sample is assessed to determine the level of expression of miRNA519c present in the nucleic acid of the subject. In general, detecting a miRNA may be carried out by determining the presence or absence of a nucleic acid containing a miRNA of interest in the test sample.

In some embodiments, hybridization methods, such as Northern analysis, or in situ hybridizations, can be used (see Current Protocols in Molecular Biology, 2012, Ausubel, F. et al., eds., John Wiley & Sons, including all supplements). For example, the presence of a miRNA associated with autoimmune disease can be indicated by hybridization to a nucleic acid probe. A “nucleic acid probe,” as used herein, can be a nucleic acid probe, such as a DNA probe or an RNA probe. For representative examples of use of nucleic acid probes, see, for example, U.S. Pat. Nos. 5,288,611 and 4,851,330.

To detect at least one miRNA of interest, a hybridization sample is formed by contacting the test sample with at least one nucleic acid probe. A probe for detecting miRNA is a labeled nucleic acid probe capable of hybridizing to miRNA. The nucleic acid probe can be, for example, a full-length nucleic acid molecule, or a portion thereof, such as an oligonucleotide of at least 10, 15, or 25 nucleotides in length and sufficient to specifically hybridize under stringent conditions to appropriate miRNA. The hybridization sample is maintained under conditions which are sufficient to allow specific hybridization of the nucleic acid probe to a miRNA target of interest. Specific hybridization can be performed under high stringency conditions or moderate stringency conditions, as appropriate. In an exemplary embodiment, the hybridization conditions for specific hybridization are high stringency. Specific hybridization, if present, is then detected using standard methods. If specific hybridization occurs between the nucleic acid probe and a miRNA in the test sample, the sequence that is present in the nucleic acid probe is also present in the miRNA of the subject. More than one nucleic acid probe can also be used concurrently in this method. Specific hybridization of any one of the nucleic acid probes is indicative of the presence of the miRNA of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising at least one miRNA of interest. Hybridization of the PNA probe to a nucleic acid sequence is indicative of the presence of a miRNA of interest.

Direct sequence analysis can also be used to detect miRNAs of interest. A sample comprising nucleic acid can be used, and PCR or other appropriate methods can be used to amplify all or a fragment of the nucleic acid, and/or its flanking sequences, if desired.

In another embodiment, arrays of oligonucleotide probes that are complementary to target nucleic acid sequences from a subject can be used to detect, identify and quantify miRNAs associated with autoimmune disease. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a sample containing miRNA is hybridized with the array and scanned for miRNAs. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein.

In brief, a target miRNA sequence is amplified by well-known amplification techniques, e.g., RT, PCR. Typically, this involves the use of primer sequences that are complementary to the target miRNA. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect miRNAs of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., 1981, Proc. Natl. Acad. Sci. USA 86:232-236), mobility shift analysis (Orita et al., 1989, Proc. Natl. Acad. Sci. USA 86:2766-2770; Rosenbaum and Reissner, 1987, Biophys. Chem. 265:1275; 1991, Keen et al., Trends Genet. 7:5); RNase protection assays (Myers, et al., 1985, Science 230:1242); Luminex xMAP™ technology; high-throughput sequencing (HTS) (Gundry and Vijg, 2011, Mutat Res, doi:10.1016/j.mrfmmm.2011.10.001); next-generation sequencing (NGS) (Voelkerding et al., 2009, Clinical Chemistry 55:641-658; Su et al., 2011, Expert Rev Mol Diagn. 11:333-343; Ji and Myllykangas, 2011, Biotechnol Genet Eng Rev 27:135-158); and/or ion semiconductor sequencing (Rusk, 2011, Nature Methods doi:10.1038/nmeth.f.330; Rothberg et al., 2011, Nature 475:348-352). These and other methods, alone or in combination, can be used to detect and quantity of at least one miRNA of interest, in a biological sample obtained from a subject. In one embodiment of the invention, the methods of assessing a biological sample to detect and quantify a miRNA of interest, as described herein, are used to diagnose, assess and characterize pathologic pregnancy or preterm labor in a subject in need thereof.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the biological sample using known techniques. Nucleic acid herein includes RNA, including mRNA, miRNA, etc. The nucleic acid can be double-stranded or single-stranded (i.e., a sense or an antisense single strand) and can be complementary to a nucleic acid encoding a polypeptide. The nucleic acid content may also be obtained from an extraction performed on a fresh or fixed biological sample.

There are many methods known in the art for the detection of specific nucleic acid sequences and new methods are continually reported. A great majority of the known specific nucleic acid detection methods utilize nucleic acid probes in specific hybridization reactions.

In the Northern blot, the nucleic acid probe may be labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. Nos. 6,159,693 and 6,270,974, and related patents. To briefly summarize one of those methods, a nucleic acid probe of at least 10 nucleotides, at least 15 nucleotides, or at least 25 nucleotides, having a sequence complementary to a desired region of the target nucleic acid of interest is hybridized in a sample, subjected to depolymerizing conditions, and the sample is treated with an ATP/luciferase system, which will luminesce if the nucleic sequence is present. In quantitative Northern blotting, levels of the polymorphic nucleic acid can be compared to wild-type levels of the nucleic acid.

A further process for the detection of hybridized nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product.

In PCR, the nucleic acid probe can be labeled with a tag as discussed before. The detection of the duplex is done using at least one primer directed to the target nucleic acid. In yet another embodiment of PCR, the detection of the hybridized duplex comprises electrophoretic gel separation followed by dye-based visualization.

Nucleic acid amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

Stem-loop RT-PCR is a PCR method that is useful in the methods of the invention to amplify and quantify miRNAs of interest (See Caifu et al., 2005, Nucleic Acids Research 33:e179; Mestdagh et al., 2008, Nucleic Acids Research 36:e143; Varkonyi-Gasic et al., 2011, Methods Mol Biol. 744:145-57). Briefly, the method includes two steps: RT and real-time PCR. First, a stem-loop RT primer is hybridized to a miRNA molecule and then reverse transcribed with a reverse transcriptase. Then, the RT products are quantified using conventional real-time PCR.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the template nucleic acid under conditions of stringency that prevent non-specific binding but permit binding of this template nucleic acid which has a significant level of homology with the probe or primer.

Among the conditions of stringency is the melting temperature (Tm) for the amplification step using the set of primers, which is in the range of about 50° C. to about 95° C. Typical hybridization and washing stringency conditions depend in part on the size (i.e., number of nucleotides in length) of the template nucleic acid or the oligonucleotide probe, the base composition and monovalent and divalent cation concentrations (Ausubel et al., 1994, eds Current Protocols in Molecular Biology).

In one embodiment, the process for determining the quantitative and qualitative profile according to the present invention is characterized in that the amplifications are real-time amplifications performed using a labeled probe, for example, a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 50° C. to 95° C. As an example, the Tm for any one of the hydrolysis-probes of the present invention is in the range of about 55° C. to about 80° C. As an example, the Tm applied for any one of the hydrolysis-probes of the present invention is about 75° C.

In another embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a nucleic acid sequence of the miRNA of interest, such as miRNA519c, and more particularly the primer includes 12 or more contiguous nucleotides substantially complementary to the sequence of the miRNA of interest. For example, a primer featured in the invention includes a nucleotide sequence sufficiently complementary to hybridize to a nucleic acid sequence of about 12 to 25 nucleotides. For example, the primer differs by no more than 1, 2, or 3 nucleotides from the target nucleotide sequence. In another aspect, the length of the primer can vary in length, for example about 15 to 28 nucleotides in length (e.g., 15, 16, 17, 18, 19, 20, 21, 22, 23, 24, 25, 26, 27 or 28 nucleotides in length).

In some embodiments, the method comprises detecting the level of one or more biomarkers associated with inflammation. In some embodiments, the method comprises detecting the level of miRNA519c in combination with detecting the level of one or more biomarkers associated with inflammation. to determine if a pregnant mother has, or is at risk for developing, pathologic pregnancy or preterm labor. Exemplary biomarkers associated with inflammation include, but are not limited to, cytokines, chemokines, acute phase reaction proteins or other miRNAs.

In some embodiments, the method comprises determining if the subject has an infection, such as a bacterial, fungal, or viral infection. For example, in certain aspects, the invention comprises determining if the subject has an infection of the genital tract, amniotic fluid, or intrauterine environment including the placenta and fetus.

### Determining Effectiveness of Therapy or Prognosis

In one aspect, the level of miRNA519c in a biological sample of a subject is used to monitor the effectiveness of treatment or the prognosis of disease. In some embodiments, the level of miRNA519c in a test sample obtained from a treated subject can be compared to the level from a reference sample obtained from that patient prior to initiation of a treatment. Clinical monitoring of treatment typically entails that each subject serve as her own baseline control. In some embodiments, test samples are obtained at multiple time points following administration of the treatment. In these embodiments, measurement of the level miRNA519c in the test samples provides an indication of the extent and duration of in vivo effect of the treatment.

Measurement of biomarker levels allow for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker levels across the samples may provide an indication as to the effectiveness of the therapy.

In one embodiment, the invention provides a method for monitoring the levels of miRNAs in response to treatment. For example, in some embodiments, the invention provides for a method of determining the efficacy of treatment in a subject, by measuring the level of miRNA519c. In one embodiment, the level of miRNA519c can be measured over time, where the level at one timepoint after the initiation of treatment is compared to the level at another timepoint after the initiation of treatment. In one embodiment, the level of miRNA519c can be measured over time, where the level at one timepoint after the initiation of treatment is compared to the level prior to the initiation of treatment.

In one embodiment, the invention provides a method for monitoring the level of miRNA519c after treatment. In one embodiment, the invention provides a method for assessing the efficacy of treatment for pathologic pregnancy or preterm labor.

For example, in one embodiment, the method indicates that the treatment is effective when the level of miRNA519c is increased in a sample of a treated subject as compared to a control diseased subject or population not receiving treatment. In one embodiment, the method indicates that the treatment is effective when the level of miRNA519c is increased in a sample of a treated subject as compared to a control sample from the subject prior to treatment. In one embodiment, the method indicates that the treatment is effective when the level miRNA519c is increased in a sample of a treated subject as compared to a sample from the subject obtained at an earlier time point during treatment.

For example, in one embodiment, the method indicates that the treatment is effective when the level of miRNA519c is decreased in a sample of a treated subject as compared to a control diseased subject or population not receiving treatment. In one embodiment, the method indicates that the treatment is effective when the level of miRNA519c is decreased in a sample of a treated subject as compared to a control sample from the subject prior to treatment. In one embodiment, the method indicates that the treatment is effective when the level miRNA519c is decreased in a sample of a treated subject as compared to a sample from the subject obtained at an earlier time point during treatment.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having an autoimmune disease can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

### Treatment Methods

The present invention provides methods for the treatment or prevention of pathologic pregnancy or preterm labor. In one embodiment, the present invention provides a method comprising detecting a differential level (e.g., a decreased level or increased level) of miRNA519c in a body sample, such as saliva, of a subject, and then administering to the subject an effective treatment. In certain embodiments, the method comprises administration of one or more therapeutic compositions including, but not limited to, prenatal steroids, neuroprotective agents, and antibiotics.

In one embodiment, the method comprises administering an agent that increases the expression or activity of miRNA519c. For example, as miRNA519c is demonstrated herein to be downregulated in pathologic pregnancy and preterm labor, then it would be desirable to increase the expression or activity of miRNA519c to normal levels using an activator as a form of therapy. In certain embodiments, the method comprises increasing the expression or activity of miRNA519c in the placenta. Activators, as used herein, are compounds that increase, open, activate, facilitate, enhance activation, sensitize, agonize, or up regulate activity of miRNA519c.

Methods and materials for increasing the expression levels of the markers of the present invention are well known and within the skill of a person in the art. A non-limiting list of known methods and materials includes: gene therapy methods, antisense oligonucleotides, antagomirs, and the like.

The invention provides a method of treating pathologic pregnancy or preterm labor by targeting the miRNAs described herein. For example, in one embodiment, the invention provides a method of treating pathologic pregnancy or preterm labor in a subject comprising administering an agent that increases the expression, activity, or level of miRNA519c. In various embodiments, the agent that increase the expression, activity, or level of miRNA519c to treat pathologic pregnancy or preterm labor is at least one of an antisense nucleic acid, a ribozyme, a miRNA, a polypeptide, an antibody, and a small molecule.

In one embodiment, the invention provides a method comprising administering to a subject an agent that increases the expression, activity, or level of miRNA519c. In various embodiments, the agent that increase the expression, activity, or level of miRNA519c to treat pathologic pregnancy or preterm labor is at least one of an antisense nucleic acid, a ribozyme, a miRNA, a polypeptide, an antibody, and a small molecule. In one embodiment, the subject is a pregnant mother. In one embodiment, the subject is a pregnant mother at risk of developing or having been diagnosed with pathologic pregnancy or preterm labor.

In one embodiment, the agent is coupled to a moiety that increases cell penetration or solubility of the agent. In one embodiment, the agent is coupled to cholesterol. In another embodiment, the agent is coupled to one or more moieties or combined with one or more compositions that are capable of directing the agent to a specific organ, tissue, or cell type. In some embodiments, the composition comprises a delivery vehicle, including but not limited to, a nanoparticle, microparticle, micelle, polymerosome, and the like, which comprises the agent. In some embodiments, the delivery vehicle is targeted to a specific treatment site, to reduce any possible systemic effects.

Modulators of miRNA

In certain embodiments, the composition comprises a modulator of one or more disease-associated miRNAs described herein. For example, in certain embodiments, the composition comprises an agent that increases the expression or activity of miRNA519, shown herein to be downregulated in pathologic pregnancy and preterm labor. In one embodiment, the composition comprises an agent that mimics the activity of miRNA519c. In one embodiment, the agent comprises miRNA519c or a mimic of miRNA519c. In one embodiment, the agent comprises a nucleic acid molecule that encodes a miRNA519c or mimic of miRNA519c.

In one embodiment, miRNA519c or mimic thereof, may be administered to a subject at risk of developing or having been diagnosed with pathologic pregnancy or preterm labor. In an exemplary embodiment, the miRNAs administered to the subject are downregulated in the disease state. In one embodiment, the miRNAs are coupled to a moiety that increases cell penetration or solubility of the miRNA. In one embodiment, the miRNA is coupled to cholesterol. In another embodiment, the miRNA is coupled to one or more moieties or combined with one or more compositions that are capable of directing the miRNA to a specific organ, tissue, or cell type. In one embodiment, the composition is administered such that the miRNA519c or mimic thereof, is directed to the placenta of the subject. In one embodiment, the composition comprising an miRNA is administered locally. In another embodiment, the composition comprising an miRNA is administered systemically.

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a miRNA519c. In certain embodiments, the oligonucleotide comprises the nucleotide sequence of miRNA519c in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3 ‘-terminus can be blocked with an aminoalkyl group. Other 3’ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of a nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleotide oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyl eneimino and methylenehydrazino backbones; sulfonate and sulfonamide backbones; amide backbones; and others having mixed N, O, S and CH2 component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference. Nucleotide oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleotide oligomer, particularly the 3′ position of the sugar on the 3′ terminal nucleotide. Nucleotide oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleotide oligomers, both the sugar and the internucleoside linkage, i.e., the backbone, are replaced with groups. Methods for making and using these nucleotide oligomers are described, for example, in “Peptide Nucleic Acids (PNA): Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

In other embodiments, a single stranded modified nucleic acid molecule (e.g., a nucleic acid molecule comprising a phosphorothioate backbone and 2′-OMe sugar modifications is conjugated to cholesterol.

A miRNA described herein, which may be in the mature or hairpin form, may be provided as a naked oligonucleotide that is capable of entering a cell. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg²⁺), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of miRNA519c. In certain embodiments, the composition comprises oligonucleotides having nucleobase identity to the nucleobase sequence of miRNA519c, and are thus designed to mimic the activity of miRNA519c. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

In one embodiment, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (2008, Current Protocols in Molecular Biology, John Wiley & Sons, New York) and as described elsewhere herein.

In order to assess the expression of the nucleic acid, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., and in Ausubel et al., and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

Vectors suitable for the insertion of the polynucleotides are vectors derived from expression vectors in prokaryotes such as pUC18, pUC19, Bluescript and the derivatives thereof, mp18, mp19, pBR322, pMB9, ColE1, pCR1, RP4, phages and “shuttle” vectors such as pSA3 and pAT28, expression vectors in yeasts such as vectors of the type of 2 micron plasmids, integration plasmids, YEP vectors, centromere plasmids and the like, expression vectors in insect cells such as vectors of the pAC series and of the pVL, expression vectors in plants such as pIBI, pEarleyGate, pAVA, pCAMBIA, pGSA, pGWB, pMDC, pMY, pORE series and the like, and expression vectors in eukaryotic cells based on viral vectors (adenoviruses, viruses associated to adenoviruses such as retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAX1, pZeoSV2, pCI, pSVL and PKSV-10, pBPV-1, pML2d and pTDT1.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al.). In a particular embodiment, the vector is a vector useful for transforming animal cells.

In one embodiment, the recombinant expression vectors may also contain nucleic acid molecules which encode a peptide or peptidomimetic modulator of the invention, described elsewhere herein.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, some advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. The promoters employed may be constitutive, tissue-specific, inducible, and/or useful under the appropriate conditions to direct high level expression of the introduced DNA segment. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue (e.g., skin). Tissue specific promoters are well known in the art and include, but are not limited to, the keratin 14 promoter and the fascin promoter sequences.

In a particular embodiment, the expression of the nucleic acid is externally controlled. In a more particular embodiment, the expression is externally controlled using the doxycycline Tet-On system.

The recombinant expression vectors may also contain a selectable marker gene which facilitates the selection of transformed or transfected host cells. Suitable selectable marker genes are genes encoding proteins such as G418 and hygromycin which confer resistance to some drugs, β-galactosidase, chloramphenicol acetyltransferase, firefly luciferase, or an immunoglobulin or portion thereof such as the Fc portion of an immunoglobulin, for example, an IgG. The selectable markers may be introduced on a separate vector from the nucleic acid of interest.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Recombinant expression vectors may be introduced into host cells to produce a recombinant cell. The cells can be prokaryotic or eukaryotic. The vector of the invention can be used to transform eukaryotic cells such as yeast cells, Saccharomyces cerevisiae, or mammal cells for example epithelial kidney 293 cells or U2OS cells, or prokaryotic cells such as bacteria, Escherichia coli or Bacillus subtilis, for example. Nucleic acid can be introduced into a cell using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Inhibitors

In some embodiments, the invention comprises an agent that inhibits miRNA519c expression or activity, which can be used in methods of treating a subject having a condition associated with an increase in miRNA519c. For example, in certain embodiments, the inhibitor comprises a nucleic acid molecule, such as an siRNA, antagomir, siRNA, shRNA, CRISPR guide RNA. In some embodiments, the inhibitor comprises a ribozyme, polypeptide, antibody, or small molecule inhibitor.

In some embodiments, the invention comprises an inhibitor of an inhibitor of miRNA519c, thereby increasing the expression or activity of miRNA519c. In some embodiments, the inhibitor comprises a nucleic acid molecule, such as an siRNA, antagomir, siRNA, shRNA, CRISPR guide RNA. In some embodiments, the inhibitor comprises a ribozyme, polypeptide, antibody, or small molecule inhibitor.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intratumoral, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents, including, for example, chemotherapeutics, immunosuppressants, corticosteroids, analgesics, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, for example, from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container. For example, such powders comprise particles wherein at least 98% of the particles by weight have a diameter greater than 0.5 nanometers and at least 95% of the particles by number have a diameter less than 7 nanometers. For example, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (e.g., having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additionally, the molecules may be delivered using a sustained-release system, such as semipermeable matrices of solid polymers containing the therapeutic agent. Various forms of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

Nucleic acids may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

In addition to the formulations described previously, the molecules may also be formulated as a depot preparation. Such long acting formulations may be administered by implantation (for example subcutaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well-known examples of delivery vehicles that may be used to deliver nucleic acids of the disclosure.

### Administration

One aspect of the invention relates to a treatment regimen for treating or preventing pathologic pregnancy or preterm labor using a composition of the invention. Compositions of the invention may be delivered alone or in combination with other compositions of the invention, and may be administered locally or systemically using appropriate methods known in the art. Administration of the compositions of the present invention to a subject may be carried out using known procedures, at dosages and for periods of time effective to prevent or treat pathologic pregnancy or preterm labor in the subject. An effective amount of the therapeutic compound necessary to achieve a therapeutic effect may vary according to factors such as the state of the disease or disorder in the subject; the age of the subject, weight of the subject, or gestational age of the fetus carried by the subject.

The regimen of administration may affect what constitutes an effective amount. Further, the dosages of the compositions may be proportionally increased or decreased as indicated by the exigencies of the therapeutic or prophylactic situation. A non-limiting example of an effective dose range for a therapeutic compound of the invention is from about 1 to about 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the compound, the age, weight, condition, general health and prior medical history of the subject being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments there between.

In one embodiment, the treatment regimen comprises daily administration of a composition of the invention. In one embodiment, a treatment regimen comprises administering a composition at least once daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year. In one embodiment, a treatment regimen comprises administering a composition two times daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year. In one embodiment, a treatment regimen comprises administering a composition three times daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year.

### Kits

The present invention also pertains to kits useful in the methods of the invention. Such kits comprise components useful in any of the methods described herein, including for example, hybridization probes or primers (e.g., labeled probes or primers), reagents for detection of labeled molecules, oligonucleotide arrays, restriction enzymes, antibodies, allele-specific oligonucleotides, means for amplification of a subject's nucleic acids, means for reverse transcribing a subject's RNA, means for analyzing a subject's nucleic acid sequence, and instructional materials. For example, in one embodiment, the kit comprises components useful for the detection and quantification of miRNA519.

The present invention also provides kits for diagnosing pathologic pregnancy or preterm labor, comprising a probe or reagent for one or more biomarkers known to be differentially expressed in during pathologic pregnancy or preterm labor.

The present invention also provides kits for diagnosing pathologic pregnancy or preterm labor, comprising a probe or reagent for one or more biomarkers known to be differentially expressed in during inflammation, such has infection-mediated inflammation. In one particular embodiment, the kit comprises reagents for quantitative amplification or detection of the selected biomarkers. Alternatively, the kit may comprise a microarray. In some embodiments the kit comprises 2 or more probes. In other embodiments, the kits may contain 3, 4, 5, 6, 7, 8, 9, 10, 15, 20, 25, or more probes.

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample.

In a further embodiment, the kit comprises the components of an assay for monitoring the effectiveness of a treatment administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator control contained in the kit, such as a positive control, a negative control, a historical control, a historical norm, or the level of another reference molecule in the biological sample. In some embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: miRNA-519c and its Role in Endotoxin Tolerance During Pregnancy

Pregnancy is characterized by a balance between immunosuppression, essential for the maintenance of semi-allogeneic fetus, and pro-inflammatory host defense, important to protect the mother and the fetus from invading organisms. Although the intrauterine setting is considered to be a protective environment for the fetus, microbes have been detected in gestational tissues and amniotic fluid without induction of significant inflammation. Adaptation to inflammatory stimulation is called endotoxin tolerance and it is identified by a decrease surge of interleukines after repeated infections. On the other hand, an excessive inflammatory response can lead to pregnancy related diseases such as PTL. To date, the exact mechanisms that contribute to the initiation and maintenance of tolerance are not completely understood. The experiments presented herein determined that dysregulation of endotoxin tolerance plays a critical role in placental inflammation. This data showed that repeated exposure of placenta to endotoxin induces a tolerant phenotype mediated by the human placenta specific miRNA-519c. It was also determined that miRNA-519c is involved in the development of endotoxin tolerance via Phosphodiesterase 3B (PDE3B) pathway. Studying human placentas from different gestational ages and from pregnancies affected by several pathologies, it was also shown that miR-519c is linked to intrauterine inflammatory pathologies. These data identify a novel mechanism of tolerance to repeated intrauterine bacterial infections essential to maintain normal pregnancy and underscore the importance of exploring the potential use of miRNA-519c as a biomarker or therapeutic option for such disorders.

To date, the exact mechanisms that contribute to the establishment and maintenance of tolerance are not completely understood. There is, however, extensive evidence suggesting the important role of miRNAs in maintenance of healthy pregnancy and their implication in endometrial receptivity, gestational tissues function and labor (Bidarimath et al., 2014, Cellular and Molecular Immunology, 11(6): 538-547; Chen et al., 2013, 253(1): 112-128; Chakrabarty et al., 2007, Proc Natl Acad Sci., 104: 15144-15149). microRNAs are a small non-coding RNA molecules that function in post-transcriptional regulation of gene expression. Human placenta tissue expresses a distinct miRNA repertoire due to the fact that a large proportion of miRNAs derive from the two largest clusters of miRNAs in humans, the chromosome 14 miRNA cluster (C14MC) and the chromosome 19 miRNA cluster (C19MC) (Morales et al., 2012, Placenta, 33: 725-734). Transcribed miRNAs can accumulate within cells or can be released into the extracellular space, including plasma and other extracellular fluids. Circulating miRNAs of placental origin are thought to derive primarily from the trophoblast layer and are present in at least two forms: vesicular miRNAs or non-vesicular, protein-bound miRNAs. (Mouillet et al., 2015, American Journal of Obstetrics and Gynecology, 213(4): S163-S172).

Extracellular vesicles (EVs) are a heterogeneous group of cell-derived membranous structures, which originate from the endosomal system or by shedding from the plasma membrane. They are present in biological fluids and are involved in multiple physiological and pathological processes (van Niel et al., 2018, Nat Rev Mol Cell Biol, 19(4): 213-228).

Here it is shown that a human placenta specific miRNA called miRNA-519c, contained inside the extracellular vesicles, plays a specific role in protecting the intra-uterine environment from uncontrolled inflammatory responses after repeated infections and therefore in controlling endotoxin tolerance. miRNA-519c belong to the C19MC family, and its members are reported to relate to pregnancy complications such as preterm birth (Miura et al., 2010, Clin Chem 56: 1767-1771); C19MC is a primate specific miRNA cluster, paternal derived (Noguer-Dance et al., 2010, Hum Mol Genet., 19(18): 3566-82). Previous studies showed that C19MC levels increase in maternal blood circulation 2 weeks after implantation and after a sharp increase phase in the first trimester, reach a plateau level for the rest of the pregnancy and decrease right after delivery (Dumont et al., 2017, Placenta, 53: 23-29). However, the role of miRNA-519c on pregnancy is largely unknown and to date there is no information on its clinical implication. The present results suggest that miRNA-519c plays a role in endotoxin tolerance via Phosphodiesterase 3B (PDE3B) pathway. PDE3B belongs to the PDE superfamily containing 11 structurally related and functionally distinct PDE gene families and hydrolyzes cAMP. Previous data has suggested that an increase of cAMP and PKA in PDE3B Knockout mice reduces TNF-α production, linking its level to endotoxin tolerance (Ahmad et al., 2016, Sci Rep, 20(6): 28056).

Several pregnancy related disorders can result in premature labor. Pre eclampsia (PEC) is a disorder of pregnancy characterized by hypertension and proteinuria whose pathogenesis is associated with an imbalance between pro-angiogenic and anti-angiogenic factors leading to incomplete transformation of the spiral arteries and resulting in hypoperfusion and ischemia of the placenta (Phipps et al., 2016, Clin J Am Soc Nephrol, 11(6): 1102-13). Another pregnancy associated pathology is premature rupture of membrane (PPROM) that complicates 2-3% of all pregnancies and, since the membranes form a barrier to ascending infection, develops most of the time in chorioamnionitis, where an inflammatory/infectious process starts at the intrauterine level often times affecting the fetus as well.

Experiments were therefore conducted to examine human placentas from different gestational ages and from pregnancies affected by different pathologies to determine whether miRNA-519c is linked to intrauterine inflammatory pathologies.

The materials and methods used in these experiments are now described.

Placental Explants

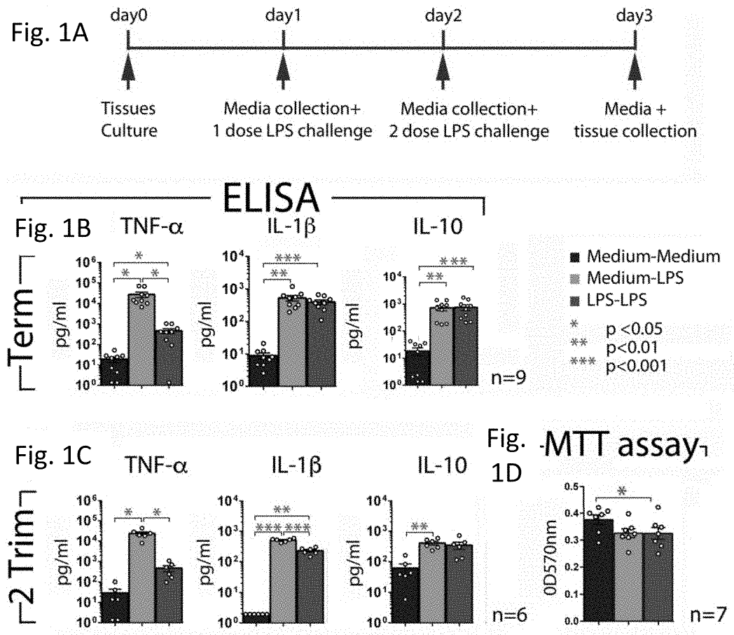

Human first and second trimester placentas from elective termination of normal pregnancies (8 to 23 weeks gestation) were collected. The second trimester placenta samples were obtained after elective terminations induced by mechanical evacuation only. Human first and second trimester and term placentas were also collected from vaginal delivery or C-section. A placental explant system was used to preserve the normal cellular architecture. Placental samples from term and third trimester were collected from the maternal side as previously described (Kim et al., 2018, Am J Reprod Immunol, December 26: e13080). Placental explants were processed immediately after collection and prepared as previously described (Peltier et al., 2013, Am J Reprod Immunol, 69: 142e149). Based on the diagram in FIG. 1A, 0.2 g of placental explants were placed in culture with 3 ml of Dulbecco's Modified Eagle Medium (DMEM) plus 10% exosomes depleted FBS (cat #EXO-FBS/HI-50A-1, SBI, Mountain View, Calif.). After 24 hours (and consequently every 24 hours for a total of 72 hours), the media was collected, centrifuged, and the supernatant was stored at −80° C. until assayed and fresh sterile media with or without LPS (3 EU/ml; Ecoli 026; B6 from Sigma Aldrich Cat # L8274) was added for a total of 2 doses (at 1 and 2 days). CytochalasinD (9.8 uM, Sigma Aldrich, St. Louis, Mo.) was added at 24 and 48 hours after the initial culture with or without LPS. Placental explants were cultured at 37° C. in a humidified incubator at 5% O₂ and CO₂ to mimic in vivo conditions at the maternal-fetal interface (Peltier et al., 2011, Am J Reprod Immunol, 66(4): 279-285) for the time of the experiment. At the end of the culture, tissue was collected and stored at −80° C. until assayed. The number of independently conducted experiments, which utilized placental explants from different donors, was as indicated in the figure legend for each experimental design. Chorioamnionitis was defined by pathology findings of chorio, PPROM by Preterm premature rupture of the membranes before 37 weeks of gestation, and PEC by BP more than 140/90 mmHg.

MTT Assay

Relative viability of the explant cultures was evaluated using 3-(4,5-dimethylthiazol-2-yl)-2,5-diphenyl tetrazolium bromide (MTT; Sigma, St. Louis, Mo.) to quantify mitochondrial dehydrogenase activity. Placental explants were cultured over 3 days as described above. MTT was added to each sample and incubated for 20 minutes to allow for crystal formation at the end of the 3 days experiment. Crystals were extracted from the tissues with isopropanol and the absorbance was measured at 562 nm.

Measurement of Cytokine and Chemokine Concentration in Placental Culture Supernatants

Supernatant cytokine concentrations were analyzed using Ready-Set-Go™ ELISA kits (eBioscience; San Diego, Calif.). The measured cytokines included TNF-a, IL-1b and IL-10 and the concentrations were expressed in pg/ml.

Real-Time PCR

Total RNA was isolated from cells with RNeasy micro Kit (Qiagen) according to the manufacturer's specifications and concentration and quality of RNA were determined spectrophotometrically at 260 nm absorbance by Nanodrop One (Thermo Scientific). Total RNA (100 ng) was reverse-transcribed using High capacity cDNA Reverse Transcription Kit, (Cat #4368814, ThermoFisher/Waltham, Mass. USA) in accordance with the manufacturer's directions. Real-time PCR was performed using Probes Master with TaqMan® Gene Expression Assays (ThermoFisher Scientific) using b-actin as normalization control and on the QuantStudio3 (Applied Biosystems). Data were analyzed using the delta-delta threshold cycle (DDCT) method (Pestana et al., 2010, Early, Rapid and Sensitive Veterinary Molecular Diagnostics—Real Time PCR Applications, Springer Science & Business Media, Dordrecht, Netherlands, pp. 247e263) and fold changes (2\hat{ }-DDCT) of mRNA expression in response to LPS-stimulation compared to control conditions were calculated. The sequence-specific oligonucleotide primers were all obtained from Thermo Fisher (Table 1).

TABLE 1
Primers used in miScript SYBR Green PCR

| MicroRNA | Assay Name | Cat number |
|---|---|---|
| RUN6-2 | Hs_RNU6-2_11 | MS00033740 |
| miR-519c-3p | Hs_miR-519c-3p_1 | MS00010003 |
| miR-543 | hsa-miR-543 | MS00010080 |
| miR-125a | hsa-miR-125a-5p | MS00003423 |
| miR-145 | hsa-miR-145-5p | MS00003528 |
| miR-29a | hsa-miR-29a-3p | MS00003262 |
| miR-29c | hsa-miR-29c-3p | MS00003269 |
| miR-19a | hsa-miR-19a-3p | MS00003192 |
| miR19b | hsa-miR-19b-3p | MS00031584 |
| miR-20a | hsa-miR-20a-5p | MS00003199 |

Primers used in TaqMan ® Gene Expression Assays

| Gene | Assay ID |
|---|---|
| GAPDH | Hs02758991_g1 |
| beta Actin | Hs01060665_g1 |
| TNF-alfa | Hs01113624_g1 |
| PDE3B | Hs00265322_m1 |
| PDE4B | Hs00277080_m1 |

microRNA Detection

Total RNA from placenta tissue was isolated using miRNeasy mini Kit (Qiagen), total RNA from conditioned media was isolated using miRNeasy serum/plasma Kit (Qiagen) and total RNA of EVs was isolated using the Total Exosome RNA and Protein Isolation Kit (Invitrogen, Carlsbad, Calif.) following the manufacturer's protocol. RNA concentrations were determined using a NanoDrop One spectrophotometer (ThermoScientific), miRNA was reverse transcribed using the miScript Reverse II RT Kit (Qiagen, Hilden, Germany) and then its expression was determined by miScript SYBR Green Primer assay (Qiagen, Hilden, Germany). Data were analyzed using the delta-delta threshold cycle (DDCT) method using RNU6-2 or miR-39-1 as internal control and fold changes (2\hat{ }-DDCT) of mRNA expression in response to LPS-stimulation compared to control conditions were calculated. In some experiments, the relative miRNA expression levels were compared by their −ddCT value as indicated in the figure legend.

miRNA Mimic Transfection and siRNA Knockdown Experiment

40 nM of miRNA mimics (mirVana, Ambion) and stealth RNAi targeting PDE3B (cat #PDE-HSS 107710, Invitrogen) were transfected using LipofectAMINE RNAiMAX (Invitrogen, Carlsbad, Calif.) according to manufacturer's directions. 24 hours after miRNA mimic transfection (and 48 hours after PDE3B siRNA transfection), cells were treated with LPS and RNA and cell culture media were collected after 3 hours (for miRNA mimic) or after 6 hrs (for PDE3B siRNA) for gene expression and cytokine secretion analysis. RNA was collected using the RNeasy micro kit (Qiagen, Germantown, Md.) and protein using ELISA as described above. RNAi GAPDH was used as a positive control to validate the transfection.

Extracellular Vesicle Isolation and Characterization

Two methods (differential ultracentrifugation and TEIR kit) were used to isolate EVs from the supernatant of placenta explants cultured after LPS or medium treatment at the indicated times to validate which method would be best when utilizing the nanosight experiment. The following experiments were done using the TEIR kit method (Invitrogen Cat #4478359).

Briefly, the differential ultracentrifugation consisted of centrifugation of the conditioned medium at 500×g (4° C.) for 10 min, 10,000×g (4° C.) for 30 min to remove cells and debris. Samples were then centrifuged at 120,000×g (4° C.) for 2- and 18 hr using TLA 100.4 rotor (ultracentrifuge Beckman Optima TLX) and pellets were finally washed with PBS and spun for an additional 20 min at 120,000×g. The resulting pellets were resuspended in 1×PBS (Helwa et al., 2017, PLoS ONE, 12(1): e0170628). With the TEIR kit, the conditioned medium was centrifuged at 500×g (4° C.) for 10 min, followed by 3,000 g for 15 min to remove cells and debris. Then the resulting conditioned medium was mixed with the TEIR reagents (Invitrogen) (2:1) according to the manufacturer's instructions. The mixture was vortexed and incubated at 4° C. overnight and then centrifuged at 10,000×g (4° C.) for 60 min to precipitate exosome pellets, then resuspended in 1×PBS. The size and distribution of the EVs were analyzed using a NanoSight LM10 instrument (NanoSight Ltd.) equipped with the nanoparticle tracking analysis (NTA) 3.2 analytic software.

In Situ Hybridization

In situ hybridization was performed as previously described (Luo et al., 2009, Biol Reprod, 81(4): 717-29). Briefly, Term normal human placenta cryosections were treated with 0.2 N HCL for 10 minutes, then the slides were fixed with 4% PFA, washed with PBS and prehybridized in hybridization (miRCURY LNA miRNA ISH buffer, Qiagen, cat #339450). After incubating for 1 hour at room temperature the prehybridization solution was replaced with hybridization buffer containing 50 nM 5′- and 3′-digoxigenin (DIG)-labeled LNA mir-591c-3p or scramble probes (Qiagen, GermanTown, Md.). The sections were hybridized for overnight at 55° C. After hybridization, the sections were washed once with 5×SSC at 55° C., twice with 1×SSC and twice with 0.2×SSC at 55° C., and once with 0.2×SSC at room temperature. Auto fluorescent was quenched with 0.3 M glycine and 0.1% Sudan Black B. Sections were blocked with 1% BSA/5% goat serum/0.1% tween-20 in PBS for 1 hour at room temperature. Sections were then incubated with antiDlG and antiPLAP antibodies (abcam. Cat #ab420 and ab198388) for overnight at 4° C. and washed 3× with PBS for 5 minutes each. The antiDlG and antiPLAP signals were visualized by incubation with secondary antibodies, goat anti-mouse IgG-Dylight 488 and goat anti-rabbit IgG-Dylight 550, respectively, (abcam, cat #ab96879 and ab96900). The slides were analyzed used a Nikon Eclipse Ti confocal microscope (Nikon, Melville, N.Y. USA).

EVs Labeling and Uptake

EVs were labeled with PKH26 based on method described by Eitan et al (2015, J Extracell Vesicles, 4: 26373) and Long et al (2017, Proc Natl Acad Sci USA, 114(17)). Briefly, EVs were mixed with 0.2 μM PKH26 (Mini26-KT, Sigma) in Diluent C. After incubating for 5 minutes, the labeling reaction was stopped by adding 1% BSA. The labeled EVs were centrifuged twice at 120,000 g for 2 hours to remove the unbounded dye. In parallel, a control tube with no EVs (dye alone) was performed to ensure lack of fluorescence uptake in the absence of EVs. The labeled EVs were resuspended in growth media supplemented with exosomes depleted FBS before added to cells. THP-1 derived macrophages were grown in 8-well chamber slides. Labeled EVs were added to the wells and allowed to incubate for 1.5 hours at 37° C. At the end of the incubation, wells were washed 4 times with PBS and fixed 20 min in formalin. Slides were blocked for 1 hour at room temperature with 1% BSA, 10 goat serum, 0.1% tween 20. After blocking, slides were incubated with anti-a-tubulin (abcam, cat #ab7291, 1:1000) for THP-1 cells, or anti-cytokerintin-7 (ThermoFisher, cat #MA5-11986, 1:200) for trophoplasts. Goat anti-mouse IgG H&L (DyLight® 488) (abcam, cat #ab96879) was used as secondary antibody. The images were obtained by Nikon Eclipse Ti confocal microscope (Nikon, Melville, N.Y. USA).

Statistical Analysis

In all figures, data are presented as mean±SEM. Statistical significance was designated at an alpha of 0.05. Graph Pad Prism Version 6.01 (GraphPad Software; San Diego, Calif.) was used for statistical analysis and graphing of results. 2-tailed Student's t test was used to compare 2 groups; 1-way ANOVA was used to compare more than 2 groups. A P value less than 0.05 was considered significant. P values and n for each experiment are provided in the figure descriptions.

The results of the experiments are now described.

### Endotoxin Tolerance is a Mechanism Present in the Placenta Tissue.

To confirm that repeated LPS doses would fail to induce a pro-inflammatory response in placental tissue, placental explants from term and preterm second trimester placentas were exposed to 2 consecutive doses of LPS given daily following the experiment design on FIG. 1A. After exposure to the first dose of LPS (FIG. 1B and FIG. 1C, green vs blue columns), in both term and preterm placenta explants, the TNF-α, IL-1β and IL-10 levels showed a statistically significant increase compared to the level after exposure only to media (Term placentas: TNF-α: 27813+/−8337 vs 19+/−5.8; 525+/−102 vs 8.9+/−1.9; IL-10: 722+/−160 vs 18.4+/−5.7. Second trimester placentas: TNF-α: 23630+/−4859 vs 28.8+/−15.6; IL-1β: 508+/−26.4 vs 1.95+/−0; IL-10: 403+/−56.7 vs 60.5+/−22.5). However, after the second LPS exposure, the TNF-a levels were statistically significantly decreased compared to the level after first LPS exposure in both, term and preterm placentas (FIG. 1B and FIG. 1C, red vs green columns. Term placentas: TNF-α: 469+/−126 vs 27813+/−8337; Second trimester placentas: TNF-α: 460+/−153 vs 23630+/−4859) In addition, IL-1β levels showed a statistically significant decrease in preterm placental explants after 2 doses of LPS compared to one dose, but at a much lower degree compared to TNF-α (FIG. 1C, red vs green column: 228+/−23.4 vs 508+/−26.4) while in the term placentas the decrease was not statistically significant (FIG. 1C, red vs green column: 398+/−60 vs 525+/−102). Finally, IL-10, an anti inflammatory cytokine, was significantly increased compared to media after both the first and the second dose of LPS in term placentas but only after the 1^{st} dose in second trimester placentas (FIG. 1B: Term Placenta: 18.4+/−5.7 vs 722+/−160 vs 753+/−186; Preterm Placenta: 60.5+/−22.5 vs 403+/−56.7 vs 342+/−91). However, there was no difference in IL-10 levels between the first and second LPS doses in both term and preterm placentas (FIG. 1B and FIG. 1C, blue vs. green vs. red column: Term Placentas: 722+/−160 vs 753+/−186. Second trimester placentas: 403+/−56.7 vs 342+/−91).

To investigate the possibility that prolonged incubation affects cytokine production through changes in viability of the cultures, mitochondrial activity of the cultures was assessed over 3 days using the MTT assay. It has been previously demonstrated that the tissue viability did not change in the first 3 days of the culture in media (13). In the current experiments, MTT analysis was performed in placental explants exposed to media, and one or two doses of LPS. There was mild (approximately 12%) but statistically significant decrease in tissue viability only after LPS treatment (either after one or two doses of LPS) compared to media. (FIG. 1D: green vs. blue columns. 0.326+/−0.016 vs 0.376+/−0.018; red vs blue column: 0.325+/−0.022 vs 0.376+/−0.018) but there was no differences in the cells viability between one or two LPS exposure (FIG. 1D: green versus red column. 0.326+/−0.016 vs 0.325+/−0.022). The relatively constant tissue viability between the nontolerazied and tolerazied cells would not explain the significant decrease in TNF-α levels (decrease by more than 98%). Moreover, IL-10 secretion remained high and did not decrease between the tollerized and the non-tollerized placenta explants. Taken together, the decrease in cytokine secretion after repeated treatments is unlikely to be caused by decreased explant viability.

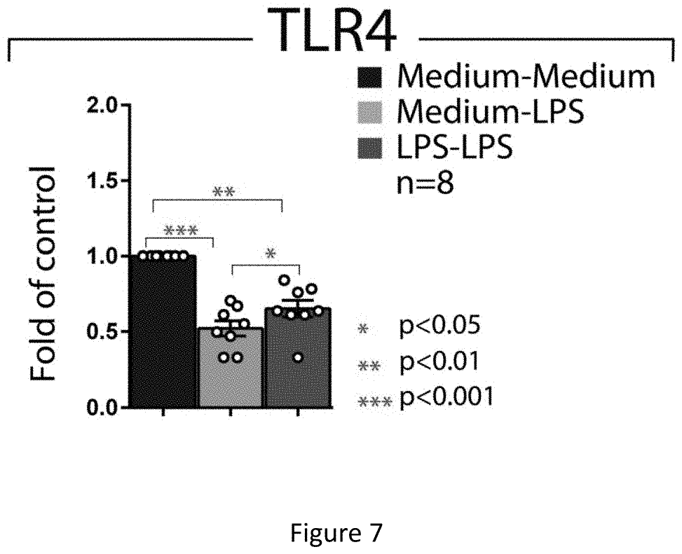

The stimulation of Toll like receptor 4 (TLR-4) by LPS increases cytokines such as TNF-a release (Lu et al., 2008, Cytokine, 42(2): 145-151). Therefore, it was investigated if decreased cytokine production after repeated LPS doses was explained by alteration of TLR-4 expression. TLR-4 expression was decreased by 50% after the first LPS dose and decreased by 35% after the second LPS dose compared to control (red column vs green column vs blue column: 0.651+/−0.056 vs 0.52+/−0.05 vs 1; FIG. 7). These data confirm that changes in TLR-4 expression after LPS treatment are not related to ET observed in placental explants after repeated LPS exposure.

### Extracellular Vesicles (EVs) are Endotoxin Tolerance's Mediators

Because of their role in intercellular communication, allowing cells to exchange proteins, lipids and genetic material, it was examined whether EVs secreted by placental tissues in the medium and their cargo were mediating ET. Extracellular vesicles were isolated by either differential ultracentrifugation (UC) according to the published procedure (Helwa et al., 2017, PLoS ONE, 12(1): e0170628) or by using the TEIR kit. The resulting extracellular vesicles pellets were subjected to size and concentration measurement by NanoSight that visualizes and analyzes particles in liquids by relating the rate of Brownian motion to particle size (Dragovic et al., 2011, Nanomedicine, 7(6): 780-8).

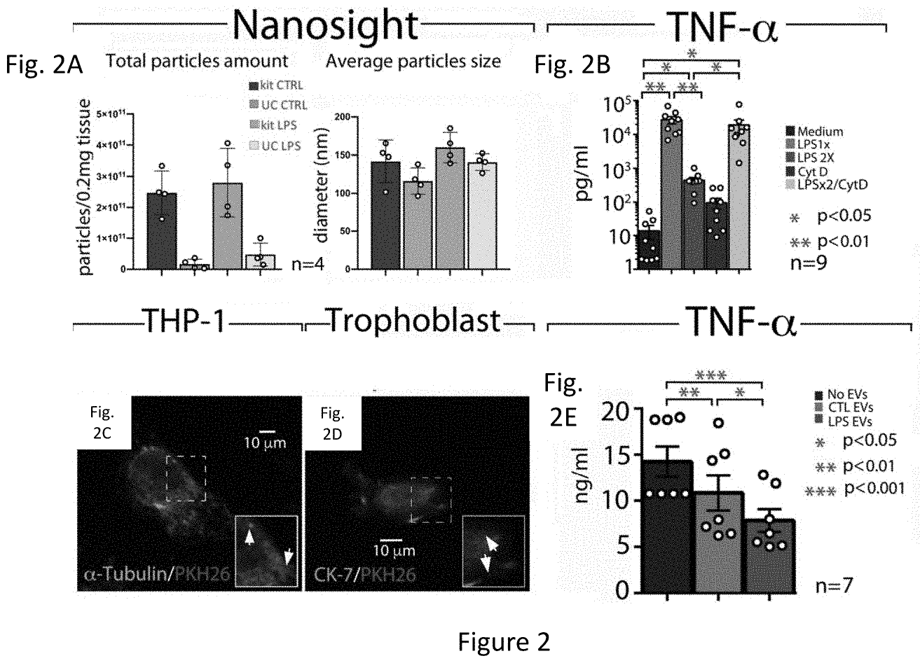

As shown in FIG. 2A, both methods allowed for the isolation of EVs that were similar in size (between 120-140 nm of diameter) with the UC isolating slightly smaller vesicles compared to the kit (Kit control: 142 nm+/−12, UC control 116.1+/−7.5 kit LPS 160+/−8.6 UC LPS 141+/−4.6); however, the TEIR kit allowed for the isolation of an increased number of EVs using a smaller amount of medium compared to the UC methods (kit control: 2.48E+/−11+/−3.05E+/−10 UC control: 1.68E¹⁰+/−6.81E⁹. kit LPS: 2.8E¹¹+/−4.77E¹⁰. Kit LPS: 2.8E¹¹+/−4.77E¹⁰, UC LPS 4.75E¹⁰+/−1.59)E¹⁰. For this reason, the kit was utilized for all of the following experiments to isolate EVs. To further verify that decreased TNF-α secretion in ET was secondary to EVs and their cargo action, term placental explants (FIG. 2B) were cultured without LPS for 24 h and the media was collected (day 1). The media was then replaced and the placental tissues were cultured for additional 24 h (day 2) with 5 different experimental conditions: with only medium (blue column), with LPS only one time (green column), with 2 LPS doses (red column), with 2 LPS doses and administration of Cytochalasin-D, a known EV uptake and production inhibitor (pink column) (35) and finally with only cytocalasin D without LPS exposure (maroon column). Media were collected and analyzed. As shown in FIG. 2B, Cytoclasin-D treatment resulted in the loss of ET expressed by increased production of TNF-α with a similar level of a not tolerized placenta despite 2 LPS doses (FIG. 2B: pink column vs green column: 18979+/−7626 vs 27051+/−6674 pg/ml), supporting the hypothesis that the secreted EVs or their cargo were responsible for TNF-α decreased level during ET. For initial extracellular vesicle uptake analysis, isolated EVs, stained with PKH26, were incubated with macrophages and primary human trophoblasts for 24 h and then followed by staining for α-tubulin (marker for macrophage) and cytokeratin 7 (marker for trophoblast). Microscopic imaging confirmed that the EVs were present externally, at the membrane level, and internally, inside the cytoplasm, in both of the cells (FIG. 2C and FIG. 2D). Finally, to further verify that the EVs were the responsible for ET, term placental explants were cultured with or without LPS as described before. Conditioned medium was collected and EVs were isolated. THP-1 cells were challenged with LPS for 24 h and co-cultured with EVs isolated from conditioned media from the tolerized or untreated placenta. THP-1 cell culture media was collected after 24 h, and TNF-α levels were analyzed by ELISA (FIG. 2E). LPS stimulation of THP-1 cell induced significant production of the pro-inflammatory cytokine TNF-α as expected (FIG. 2E E, blue column: 14.2+/−1.65 ng/ml). However, there was a significant reduction in TNF-α production in LPS-stimulated THP-1 cells co-cultured with EVs from tolerized placental explants and from untreated placental explants (FIG. 2E, red vs green columns: 7.83+/−1.23 vs 10.8+/−1.8 ng/ml). This indicates that repeated LPS exposure of the placenta is likely to induce tolerance via production of EVs whose cargo is able of eliciting anti-inflammatory effects. Collectively, these data suggest that placental EVs contain a component that can program ET at the level of the macrophage and the trophoblast.

Extracellular Vesicles Contain Several microRNA Including miRNA-519c, Possible Mediator of Endotoxin Tolerance.

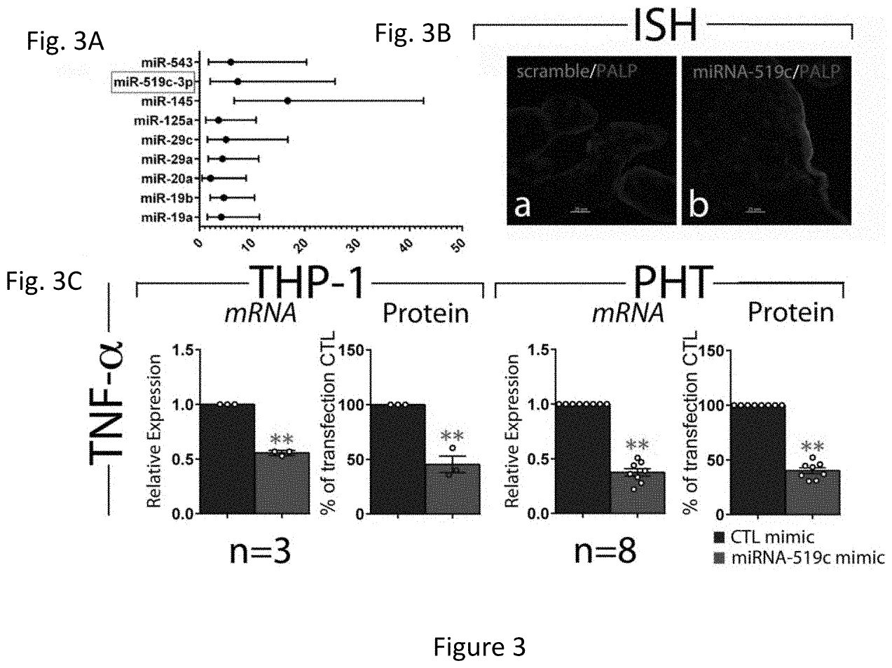

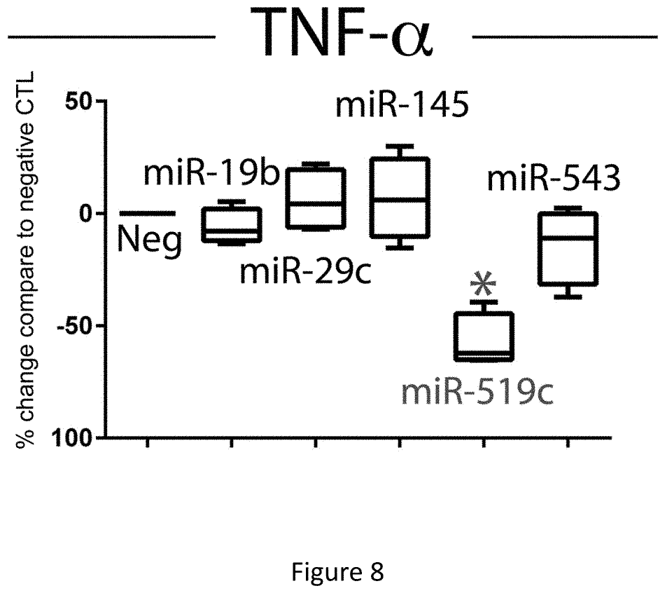

Recently, mRNAs and microRNAs (miRNAs) have been identified inside extracellular vesicles, which can be taken up by neighboring or distant cells and subsequently modulate recipient cell's functions. It was therefore examined whether LPS was stimulating placental trophoblasts to produce specific miRNAs packaged within EVs and playing a role during ET. Profile miRNAs screen array was performed on EVs from tolerized placental explants. Nine miRNAs in EVs were found to be significantly induced by LPS treated placenta (FIG. 3A). To investigate which miRNA in placental EVs was responsible for ET, various miRNA mimics were transfected in THP-1 cells (FIG. 8). Compared to other miRNAs, miRNA-519c was the only miRNA which significantly decreased TNF-α gene expression, suggesting it to be one of the effectors mediating placental ET. Consistent with the real-time PCR data, in situ hybridization for miRNA519c confirmed the localization of the microRNA in the trophoblast labeled with PALP (FIG. 3B). Finally, to further verify its function, miRNA-519c mimic and control mimic were transfected into THP-1 cells and primary trophoblast cells and the cells were challenged with LPS 24 hours later. As shown in FIG. 3C, miRNA-519c mimic transfection statistically significantly decreased TNF-α gene expression and protein production in both cells types compared to control mimic transfection (THP-1 cells: INF-α miRNA level: 0.557+/−0.013 vs 1, p<0.01; TNF-α protein: 45.4+/−7.7 vs 100, p<0.01 N=3. Trophoblast cells: INF-α miRNA level: 0.375+/−0.035, p<0.01; TNF-α protein: 40.1+/−2.7, p<0.01 n=8). These results suggest that placenta-specific miRNA-519c packaged within placental EVs can be responsible for ET.

miRNA-519c Gets Transported to the Target Cells Inside the Extracellular Vesicles

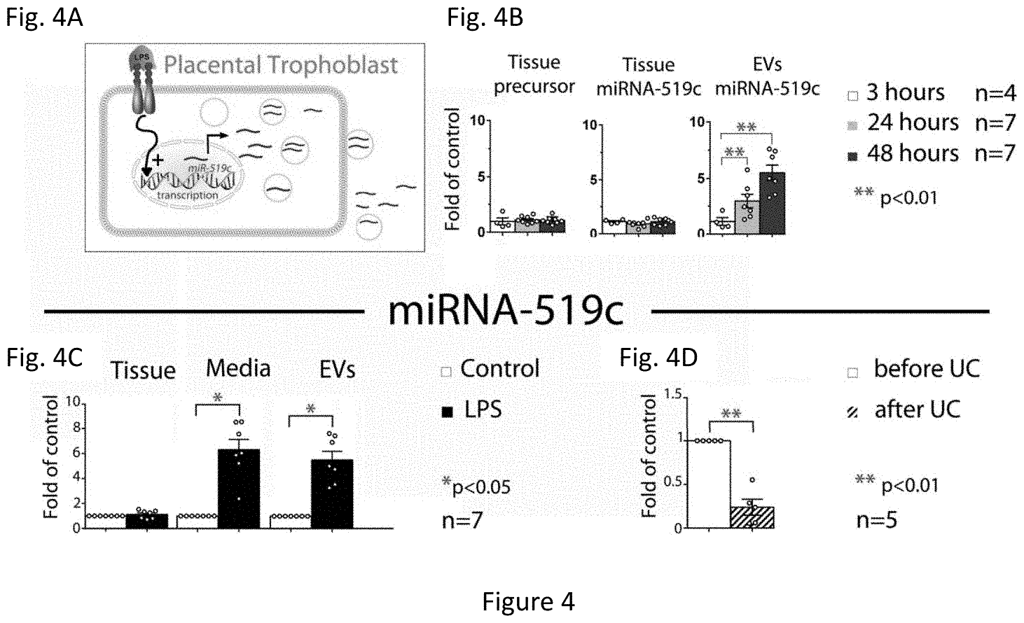

Studies were conducted to examine whether miRNA-519c is produced inside the trophoblast and whether it is transported extracellularly packaged in EVs or secreted as free miRNA (FIG. 4A). For example, if EVs packed miRNA-519c, then, they may induce tolerance in target cells either at the maternal-fetal interface or in other organs after being transported in the maternal circulation. To determine how miRNA-519c was secreted, it was investigated whether after LPS there was an increase of the miRNA-519c precursor or the mature form. The placenta explants culture tissue were stimulated with LPS. At 3, 24 and 48 h tissue and medium were collected, and EVs were isolated from media. miRNA-519c level (precursor and mature form) were determined by qPCR and expressed as fold change compared to untreated control. As demonstrated in FIG. 4B, after LPS treatment, neither the precursor or the mature miRNA-519c were increased in the tissue; but the mature form was statistically significantly increased in EVs (FIG. 4B: Tissue precursor: 3 hours 0.965+/−0.316, 24 hours 1.15+/−0.128, 48 hours 1.13+/−0.109. Cellular miRNA-519c: 3 hours 1.13+/−0.09, 24 hours 0.909+/−0.122, 48 hours 1.12+/−0.124. EVs miRNA-519c: 3 hours 1.13+/−0.346, 24 hours 2.96+/−0.598, 48 hours 5.51+/−0.687). This indicates that miRNA-519c is produced in the trophoblast, not stored intracellularly, but continuously transported outside the cells.

To confirm then that miRNA-519c was secreted into the media, its level in the tissue, in the total media and inside the EVs was detected after LPS exposure (FIG. 4C). It was found that its level was statistically significantly increased in the media and inside the EVs, but not inside the tissue (Tissue: 1.12+/−0.124, Media 6.31+/−0.825 p<0.05, EVs 5.51+/−0.687, p<0.05) supporting furthermore the concept that miRNA-519c is not stored intracellularly. To study whether miRNA-519c was secreted as free form or vesicular form, the media was then ultracentrifugated at 180 g for 18 hours (FIG. 4D) to deplete the EVs from the media and the miRNA-519c level was detected inside the media. It was found that miRNA-519c levels decreased by around 70% demonstrating that it was mainly secreted inside the EVs than as free form (0.238+/−0.091, p<0.01). Taken together, these data suggest that miRNA-519c is not stored intracellularly, but it is secreted inside the EVs to be transported to the target tissue.

miRNA-519c Mediates Endotoxin Tolerance Via PDE3B

To study the functional consequences of miRNA-519c induction by LPS predicted mRNA target sites were searched for using miRBase (Kozomara et al., 2014, NAR, 42: D68-D73; Kozomara et al., 2011, NAR, 39: D152-1527; Griffiths-Jones et al, 2008, NAR, 36: D154-158; Griffiths-Jones et al., 2006, NAR, 34: D140-D144; Griffiths-Jones, 2004, NAR, 32: D109-D111). The 3′UTRs of mRNA coding for PDE3B was found to contain miRNA-519c target sequences. Recently, it was reported that a phosphodiesterase inhibitor Pentoxixyline (PTX) inhibited LPS induced inflammation and TNF-α production among other pro-inflammatory cytokines in the human placenta with relative preservation of the anti-inflammatory mediators level (Speer et al., 2017, Placenta, Oct:58: 60-66). The anti-inflammatory effects of PTX were observed independently of the timing of LPS administration. Other reports suggested that ablation of PDE3B reduced LPS-induced TNF-α in white fat tissue (Ahmad et al., 2016, Sci Rep, 20(6): 28056) and that PDE3B is a novel target for TNF-α regulation (Zhang et al., 2002, Diabetes, 51(10): 2929-2935).

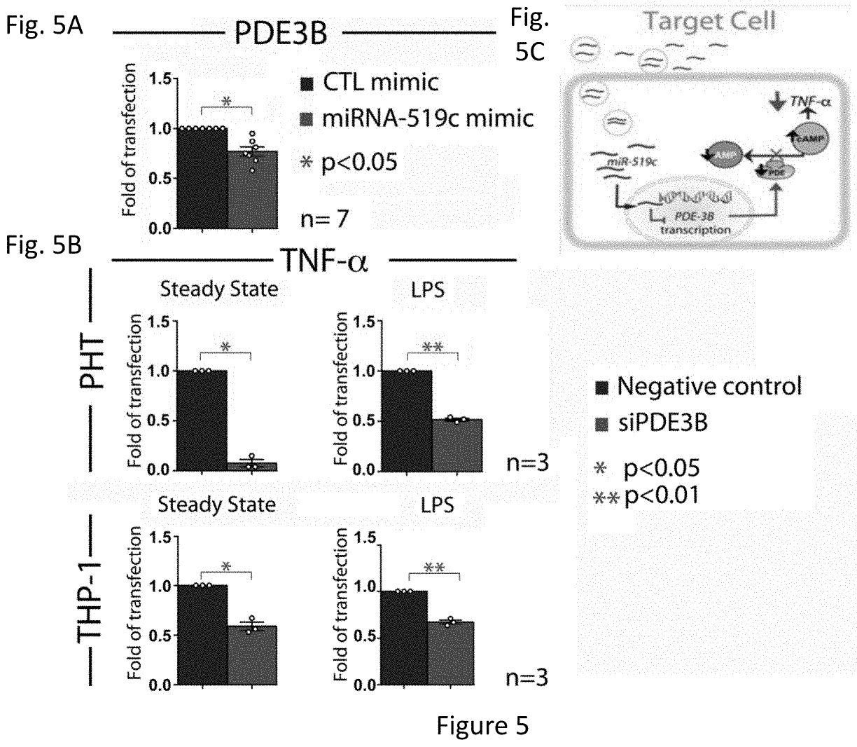

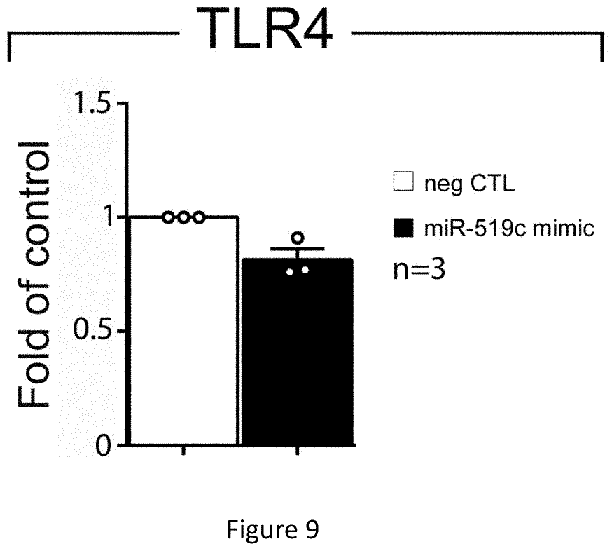

Therefore, experiments were conducted to examine the role of phosphodiesterase 3B, belonging to the phosphodiesterase family. Transfection of miRNA-519c mimic reduced PDE3B level in trophoblast cells (FIG. 5A: PDE3B 0.767+/−0.046, p<0.05). To further confirm this data, siPDE3B was transfected in PTH and THP-1 cells and TNF-α mRNA level was then detected by qPCR in steady state (without LPS) or during inflammatory state (after LPS exposure). In trophoblast cells, TNF-α expression was decreased during inflammation as well as in normal conditions (FIG. 5B: with LPS 0.517+/−0.015 p<0.01 vs steady state 0.077+/−0.037 p<0.05). On THP-1 cells, as well, lack of PDB3B decreased TNF-α production (FIG. 5B steady state: 0.589+/−0.042, p<0.05 n=3, with LPS: 0.670+/−0.021, p<0.01, n=3). These data demonstrate that PDE3B affected TNF-α production by both types of cells and especially during an inflammatory status. TLR4 is a toll like receptor known for recognizing LPS and for starting inflammatory reaction via the intracellular signaling pathway NF-κB. To confirm that miRNA-519c was controlling TNF-α level via PDE3B pathway and not via TLR-4 activation, it was confirmed that transfected miRNA-519c did not alter the TLR4 gene expression in THP-1 (FIG. 9: 0.813+/−0.048) verifying its role in a downstream pathway. Taken together, these data demonstrate that miRNA-519c, via its downstream target PDE3B, is involved in TNF-α downregulation and subsequently in endotoxin tolerance (FIG. 5C).

miRNA-519c Levels are Stable During Pregnancy but Decreased in Mothers with Inflammatory Processes.

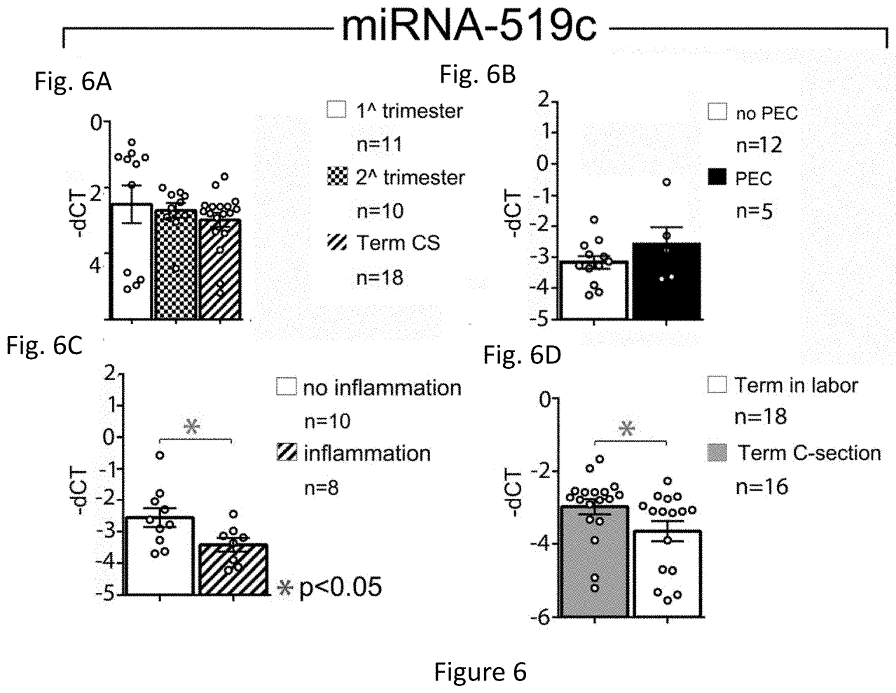

To track miRNA-519c expression in maternal placenta tissue during human pregnancy, placentas from first and second trimester elective terminations and from women not in labor and who underwent a c-section delivery were studied. miRNA-519c levels were examined in the placenta tissue: miRNA-519c levels were stable throughout the pregnancy (FIG. 6A: first trimester −2.5+/−0.571, second trimester −2.69+/−0.226, term CS −2.98+/−0.214). To examine whether miRNA-519c is connected with inflammation, placentas were studied from women affected by different pathologies. Placentas from women affected by PEC (not in labor) did not show a statistically significant difference in miRNA-519c level compared to women who did not present any features of this disorder (FIG. 6B: −3.17+/−0.203 vs −2.6+/−0.571). However, analyzing placentas from women not in labor and affected by premature rupture of membrane (PPROM) or chorioamnionitis, both processes connected with an uncontrolled inflammation, miRNA-519c levels were statistically significantly decreased compared to placentas from women not affected by these 2 pathologies (FIG. 6C: −3.4+/−0.297 vs −2.56+/−0.299 p<0.05). Moreover, it is well documented that uterine inflammation in the absence of infections is a labor-related physiologic phenomenon and a critical element in the initiation of labor (Shynlova et al., 2013, Reprod Sci, 20(2): 154-67). Therefore, to further examine whether decreased miRNA-519c levels are associated with an inflammatory process, its level was detected in women in labor and compared them to the ones in women who delivered via CS and without being in labor. As presented in FIG. 6D, laboring women presented a lower miRNA519c level compared to women who delivered via CS (FIG. 6D: −3.65+/−0.275 vs −2.98+/−0.214). These data confirm the principal role of miRNA-519c in decreasing the inflammatory process at the level of the placenta and suggests that lack of miRNA-519c places women at risk of developing inflammation related pathologies such as PPROM or chorioamnionitis or to go into labor.

Endotoxin Tolerance was Mediated in Part by the Placenta-Specific miRNA-519c

miRNAs play important roles in pregnancy, with a wide range of miRNAs implicated in implantation and labor (Ouyang et al., 2014, Placenta, 35 Suppl: 69) as well as in pathologic pregnancy conditions (Thamotharan et al., 2017, PLoS One, 12(5); Anton et al., 2015, PLoS One, 10(3)). Cells secrete circulating miRNAs in to the extracellular environment to mediate cell-cell signaling (Cortez et al., 2011, Nat Rev Clin Oncol, 8(8): 467-477). The human placenta has both an increased average miRNA copy number compared to other tissues as well as a unique signature of miRNA expression (Liang et al., 2007, BMC Genomics, 8: 166) suggesting an important role in placental function. A group of miRNAs encoded by the human chromosome 19, called Chromosome 19 miRNA Cluster (C19MC), is found only in primates and is expressed almost exclusively in the placenta (Donker et al., 2012, Mol Hum Reprod, 18: 417-424). The trophoblast layer releases C19MC miRNAs into maternal circulation (Donker et al., 2012, Mol Hum Reprod, 18: 417-424; Chim et al., 2008, Clin Chem, 54: 482-490) making them available to affect maternal physiology through regulation of gene expression in maternal tissues. C19MC miRNAs have also been detected in the amniotic fluid and in the fetal circulation (Chang et al., 2017, FASEB J, 31(7): 2760-2770). Recent investigations began to uncover the function of trophoblastic C19MC miRNAs: members of this cluster are involved in implantation and migration of extravillous trophoblasts (Xie et al., 2014, Endocrinology, 155: 4975-4985) and viral infection (Delorme-Axford et al., 2013, Proc Natl Acad Sci USA, 110(29): 12048-53). The level of C19MC miRNAs in maternal blood increases from the second half of the first trimester of pregnancy as early as 5 weeks after implantation (Dumont et al., 2017, Placenta, 53: 23-29) to term, and then rapidly declines following delivery (Gilad et al., 2008, PLoS One, 3: e3148; Miura et al., 2010, Clin Chem, 56: 1767-1771). This data showed that miRNA-519c levels specifically remain stable in the placental tissue from 8 weeks until the end of pregnancy in agreement with the findings by Dumont et al (2017, Placenta, 53: 23-29). While not wishing to be bound by any particular theory, it is speculated that miRNA-519c is needed in the amniotic environment to allow the semiallogenic fetal tissue to differentiate without inducing any immunological reaction as well as to protect it from infection.

Although many pregnant women are exposed to repeated infections (DiGiulio et al., 2010, Am J Reprod Immunol, 64(1): 38-57), a high rate of pregnancies can also overcome genital tract and amniotic fluid infections and only 2-4% of pregnant women develop infections (Kourtis et al., 2014, N Eng J med, 370(23): 2211-2218). Moreover, adaptation to inflammatory stimulation may be critical in preventing rejection of the semi-allogenic fetus by excessive maternal inflammatory responses to infectious agents (Blackburn and Loper, 1992, Maternal, fetal, and neonatal physiology: a clinical perspective. WB Saunders; Philadelphia). Bacterial infections are associated with adverse pregnancy outcomes such as IUGR or preterm delivery due to failure to display attenuation of inflammatory responses (Murphy et al., 2009, American Journal of Obstetrics and Gynecology, 200(3): 308).

Here, it is demonstrated that repeated human placental exposure to endotoxin challenges creates a tolerant maternal (or placental phenotype) called immuno tolerance, where the inflammatory response is reduced due to the repeated exposure to endotoxins. This process already described in several clinical settings (Lopez-Collazo et al, 2013, Crit Care, 17(6): 242) was never clearly described in placental tissue showing its pivotal role in keeping an ongoing healthy pregnancy. It has been previously demonstrated that endotoxin tolerance occurred after both low and high LPS doses (Kim et al., 2018, Am J Reprod Immuno, December 26:e13080), but in the present experiments, a low dose was used to mimic infection seen in clinical chorioamnionitis and have a better clinical correlation.

Previous results demonstrated that placental cultures were able to mount a robust inflammatory response after an extended period in culture. Placental explants were exposed to LPS after 3 days of incubation in LPS-free media. LPS exposure on day 3 was able to stimulate high levels of TNF-α and IL-10 secretion (Kim et al., 2018, Am J Reprod Immuno, December 26:e13080). These data show that after 3 days in culture the placental tissue is still able to respond to infectious stimuli. Along with the results of the MTT assay presented with this work, the data confirm that placental tissue remains viable despite 3 days incubation times.

Based on the present results, endotoxin tolerance was mediated in part by the placenta-specific miRNA-519c, which belong to the Chromosome 19 miRNA cluster and protects the maternal-fetal interface from the exaggerated inflammatory response seen in infection-induced preterm labor. Until now, miRNA-519c was reported being involved in several types of cancer including testicular and prostate cancer (Flor et al., 2016, Cancer Genomics Proteomics, 13(4): 281-9). Placentas from women affected by PPROM and/or chorio (confirmed by placenta pathology) showed decreased levels of miRNA5-19c compared to placentas from women not affected by inflammatory disease, confirming the pivotal role of this microRNA in inducing ET and in decreasing inflammation. Moreover, women in labor showed a decreased level of miRNA-519c compared to women delivering via CS, further verifying its role in suppressing inflammatory response. Placentas from women affected by other diseases not secondary to inflammation/infection such as PEC (where the pathogenesis is thought to be secondary to a vascular disease) did not show a different miRNA-519c levels compared to normal control, proving again that miRNA-519c is only involved in inflammatory processes (FIG. 6B and FIG. 6C). This is the first report investigating the pivotal role of miRNA-519c on inflammation in placenta tissues. Based on this data, trophoblast cells produce and secrete miRNA-519c in EVs and it is mainly localized inside the extracellular vesicles, allowing an increased stability and longevity. These data are consistent with an important role in regulating extracellular process involving different tissue and cell types.

miRNA-519c controls TNF-α secretion, the major regulator of endotoxin tolerance, via the PDE3B pathway, known to affect intracellular cAMP. It has been previously reported that a PDEs inhibitor, pentoxifylline (PTX), inhibits LPS induced TNF-α, among other pro-inflammatory cytokines in human placenta with the relative preservation of anti-inflammatory mediator levels (Speer et al., 2017, Placenta, 58: 60-66). The anti-inflammatory effects of PTX were observed independently of the timing of LPS administration (before, during or after LPS). Studies have suggested that ablation of PDE3B reduced LPS-induced TNF-α secretion in white fat tissue (Ahmad et al., 2016, Sci Rep, 6: 28056) and therefore suggest PDE3B as a possible new target for TNF-α regulation (Zhang et al., 2002, Diabetes, 51(10): 2929-2935). These findings support the hypothesis that PDE3B plays a role in ET and is a downstream target of miRNA-519c.

Finally, miRNA-519c mimic transfection in THP-1 cells did not alter TLR4 gene expression suggesting that TNF-α decreased levels were not due to a decreased TLR4 level but due to a mechanism downstream the receptor. Moreover, the increased level of TLR4 after 2 doses of LPS can be explained by a negative loop that TNF-α exerts on TLR4 level (Tsai et al., 2009, Shock 32(1): 40-48).

In conclusion, endotoxin tolerance is present in placental tissue and is a mechanism that is involved in protecting both mother and fetus from different pregnancy related inflammatory diseases such as PPROM or chorioamnionitis (that ultimately lead to preterm labor) or from the actual onset of labor. miRNA-519c is responsible for the decreased TNF-α level after multiple endotoxin exposure and this is the first report regarding the role of this microRNA in endotoxin tolerance. miRNA-519c gets packed inside extracellular vesicles and transported to different type of cells. This leads to a decrease of PDE3B and subsequent a decrease of TNF-α level. The presented data are highly significant since it addresses preterm deliveries, the leading causes of neonatal mortality and morbidity affecting half a million pregnancies/year.

### Example 2: Biomarkers from Minimally Invasive Sampling Reflective of the Placental Immune Microenvironment

Placental miRNAs play an essential role in maintaining a balanced immune environment, conducive to a healthy pregnancy. The majority of placental miRNAs are located intracellularly. However, placental cells can selectively secrete miRNAs to the extracellular space mainly packaged within circulating extracellular vesicles (EVs), which act as vehicles to transport miRNA to distant tissues. As described above, the placenta-specific miR-519c is a key anti-inflammatory miRNA regulating placental immune tolerance involved in the pathogenesis of preterm labor. Identifying biomarkers in a non-invasive biofluid that can reflect the placental immune microenvironment has not yet been identified.

The presently described experiments were conducted to identify if saliva's EVs samples from pregnant women will contain the placenta specific miR-519c and if its levels correlate with that in the placental.

Maternal plasma, amniotic fluid, saliva, and placental tissues were collected simultaneously during term normal cesarean section deliveries (n=8). EVs were isolated using differential ultracentrifugation method from these biofluids. EVs miR-519c content were compared to placental tissue's miR-519c. RNA was reverse-transcribed using the qScript microRNA cDNA Synthesis kit and the miRNA expression was determined by Real-Time PCR using PerfeCta SYBR Green master mix. The relative miRNA expression levels were expressed as dCT value using RNU-6 (placenta tissue and saliva EVs) and miR-39-1 (plasma and amniotic fluid) as the internal control. Correlation in the various fluid compartments was determined using Spearman correlation with P<0.05 required for significance.

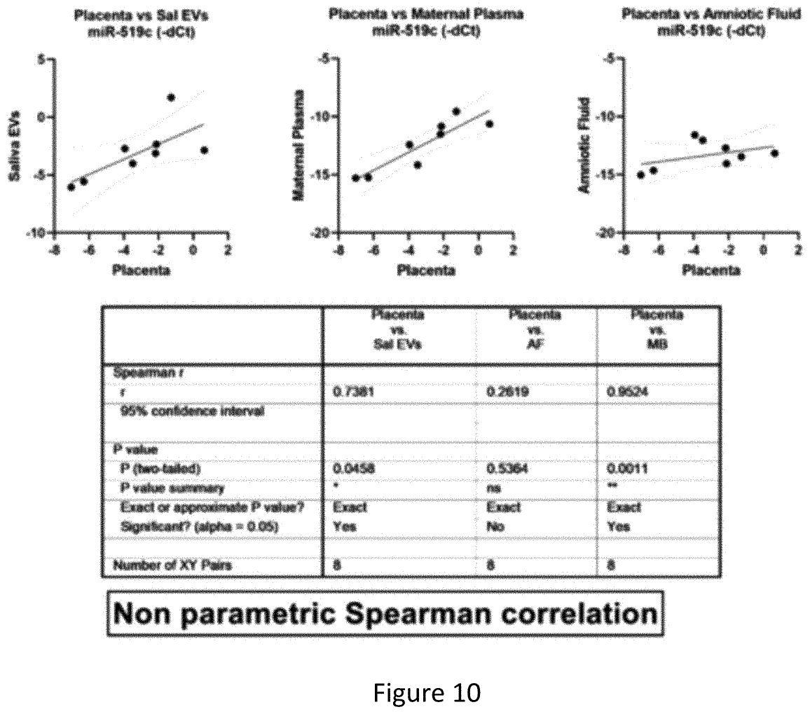

miR-519c was detected in EVs from maternal plasma, amniotic fluid, and saliva. miR-519c had comparable expression in maternal plasma and amniotic fluid, and surprisingly higher expression in the saliva samples. Furthermore, EVs miR-519c expression was strongly correlated between the placenta and maternal plasma samples (n=8, Spearman r=0.96, p-value=0.0011), as well as between the placenta and saliva samples (n=8, Spearman r=0.74, p-value=0.046). However, there was no correlation between the placenta and amniotic fluid samples (FIG. 10).

This is the first study to demonstrate that a placenta-specific miRNA can be detected in the saliva of pregnant women that correlates with the placental miRNA levels. Thus, saliva miRNAs can be useful biomarkers to reflect placental immune status in pathologic pregnancy such as preterm birth.

### Example 3: Placental Samples from Asymptomatic COVID Positive Compared to COVID Negative Mothers

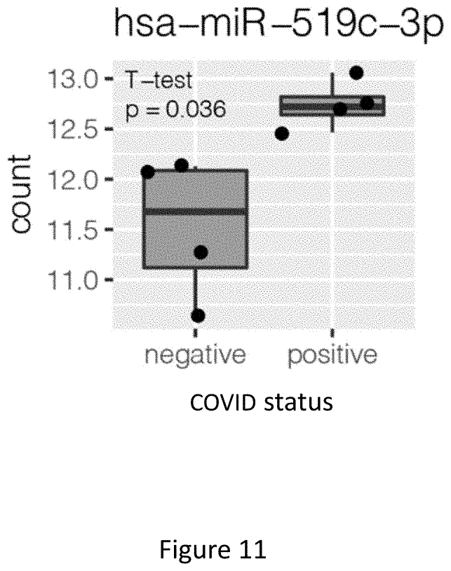

Experiments were conducted to examine the level of miR-519c in placental tissue in pregnant mothers having, or not having, SARS-CoV2 (i.e., COVID-19) infection. Sequence analysis was performed for both placental mRNAs and microRNAs. Surprisingly, it was observed that COVID-19 infection significantly upregulates miR-519c (FIG. 11), a protective miRNA against infection-induced preterm labor (as demonstrated in the above examples). As demonstrated above, a decrease in placental miR-519c makes the placenta more susceptible to bacterial infection and preterm labor. However, COVID-19 unexpectedly increased miR-519c, which will protect the pregnancy from bacterial infection and preterm labor. This data indicates that COVID-19 may be protective against infection-induced preterm labor.

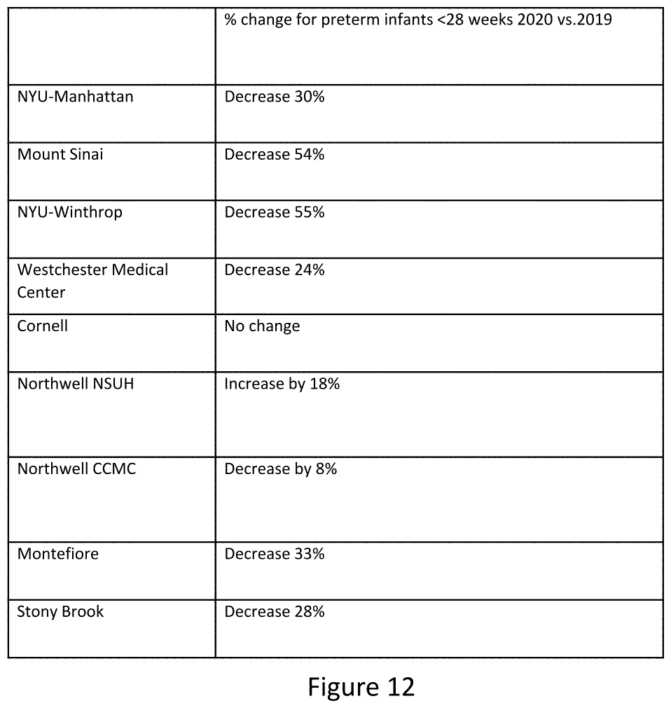

Further, a survey of NICUs in the New York city area showed that was a decrease in the number of preterm infants (<28 weeks gestation) in most centers (FIG. 12). Together this data further demonstrates that miRNA-519c is protective against infection-induced preterm labor.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for diagnosing a subject as having, or being at risk for having, pathologic pregnancy comprising:
a. measuring the level of miRNA519c in a biological sample from the subject; and
b. comparing the level of miRNA519c in the biological sample from the subject to the level of miRNA519c in a comparator, wherein a differential level of miRNA519c in the biological sample relative to the comparator indicates that the subject has, or is at risk for developing, pathologic pregnancy.

2. The method of claim 1, wherein the pathologic pregnancy is associated with the pathophysiology of inflammation.

3. The method of claim 1, wherein the pathologic pregnancy is preterm premature rupture of membrane (PPROM), preterm labor, preeclampsia, intrauterine growth restriction, placental abruption, chromosomal anomalies or chorioamnionitis.

4. The method of claim 1, wherein the biological sample is at least one selected from the group consisting of saliva, urine, blood, serum, and plasma.

5. The method of claim 1, wherein the measuring the level of miRNA519c in the biological sample from the subject comprises at least one technique selected from the group consisting of reverse transcription, polymerase chain reaction (PCR), and microarray analysis.

6. The method of claim 1, wherein the method comprises measuring the level of miRNA519c in extracellular vesicles (EVs) isolated from the biological sample.

7. The method of claim 1, further comprising administering to the subject a therapeutic agent to treat or prevent pathologic pregnancy.

8. The method of claim 1, further comprising measuring an increased level of one or more biomarkers associated with inflammation in the subject relative to a comparator.

9. The method of claim 8, wherein the measurement of an increased level of one or more biomarkers associated with inflammation and the measurement of a differential level of miRNA519c indicates that the subject will proceed to delivery of the fetus or other pathologic pregnancy outcome.

10. The method of claim 9, wherein the method comprises administration to the subject of a specific therapy selected from: prenatal steroids, neuroprotective agents, and antibiotics.

11. A method of treating or preventing pathologic pregnancy in a subject comprising
a. measuring the level of miRNA519c in a biological sample from the subject;
b. comparing the level of miRNA519c in the biological sample from the subject to the level of miRNA519c in a comparator, wherein a differential level of miRNA519c in the biological sample relative to the comparator indicates that the subject has, or is at risk for developing, pathologic pregnancy; and
c. administering to the subject a therapeutic agent to treat or prevent pathologic pregnancy.

12. The method of claim 10, wherein the therapeutic agent comprises an immunosuppressive agent.

13. The method of claim 10, wherein the therapeutic agent comprises an agent that increases or decreases the expression or activity of miRNA519c in the placenta of the subject.

14. The method of claim 12, wherein the therapeutic agent comprises miRNA519c or a miRNA519c mimic.

15. The method of claim 12, wherein the therapeutic agent comprises a nucleic acid molecule encoding miRNA519c or a miRNA519c mimic.

16. A method of preparing a sample comprising,
a. providing a biological sample from a subject;
b. isolating extracellular vesicles from the biological sample;
c. selectively extracting RNA from the isolated extracellular vesicles; and
d. performing an amplification reaction on the extracted RNA to detect the level of miRNA519c in the biological sample, wherein the amplification reaction is performed using primers specific for miRNA519c.
